SEQUENCE LISTING (84 sequences)

SEQ ID NO: 1 - DNA, 4200 bp, Homo sapiens
acagcattaacatttagtgggagtgcagtgagaattgggtttaacttctggcatttctgggcttgtggcttgtggttgattttttatttactttgcaaaagtttctgataggcggagcatctagtttcaacttccttttgcagcaagttctttcctgcactaatcacaattcttggaagaggagaactggacgttgtgaacagagttagctggtaaatgtcctcttaaaagatccaaaaaatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattacatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatagaatcaatcataaagtgcacacctttattcagaactttagtattaaatcagttctcaatttcattttttatgtattgttttactcctttttattcatacgtaaaattttggattaatttgtgaaaatgtaattataagctgagaccggtggctctcttcttaaaagcaccatattaaatcctggaaaactaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 3 - DNA, 1114 bp, Homo sapiens
acactgcgctgcccagaggccccgctgactcctgccagcctccaggtccccgtggtaccaaagctgaacatggacgtgaccatccagcacccctggttcaagcgcaccctggggcccttctaccccagccggctgttcgaccagtttttcggcgagggcctttttgagtatgacctgctgcccttcctgtcgtccaccatcagcccctactaccgccagtccctcttccgcaccgtgctggactccggcatctctgaggttcgatccgaccgggacaagttcgtcatcttcctcgatgtgaagcacttctccccggaggacctcaccgtgaaggtgcaggacgactttgtggagatccacggaaagcacaacgagcgccaggacgaccacggctacatttcccgtgagttccaccgccgctaccgcctgccgtccaacgtggaccagtcggccctctcttgctccctgtctgccgatggcatgctgaccttctgtggccccaagatccagactggcctggatgccacccacgccgagcgagccatccccgtgtcgcgggaggagaagcccacctcggctccctcgtcctaagcaggcattgcctcggctggctcccctgcagccctggcccatcatggggggagcaccctgagggcggggtgtctgtcttcctttgcttcccttttttcctttccaccttctcacatggaatgagggtttgagagagcagccaggagagcttagggtctcagggtgtcccagaccccgacaccggccagtggcggaagtgaccgcacctcacactcctttagatagcagcctggctcccctggggtgcaggcgcctcaactctgctgagggtccagaaggagggggtgacctccggccaggtgcctcctgacacacctgcagcctccctccgcggcgggccctgcccacacctcctggggcgcgtgaggcccgtggggccggggcttctgtgcacctgggctctcgcggcctcttctctcagaccgtcttcctccaacccctctatgtagtgccgctcttggggacatgggtcgcccatgagagcgcagcccgcggcaatcaataaacagcaggtgatacaagc

SEQ ID NO: 4 - protein, 173 aa, Homo sapiens
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 5 - DNA, 691 bp, Homo sapiens
gacccctcacactcacctagccaccatggacatcgccatccaccacccctggatccgccgccccttctttcctttccactcccccagccgcctctttgaccagttcttcggagagcacctgttggagtctgatcttttcccgacgtctacttccctgagtcccttctaccttcggccaccctccttcctgcgggcacccagctggtttgacactggactctcagagatgcgcctggagaaggacaggttctctgtcaacctggatgtgaagcacttctccccagaggaactcaaagttaaggtgttgggagatgtgattgaggtgcatggaaaacatgaagagcgccaggatgaacatggtttcatctccagggagttccacaggaaataccggatcccagctgatgtagaccctctcaccattacttcatccctgtcatctgatggggtcctcactgtgaatggaccaaggaaacaggtctctggccctgagcgcaccattcccatcacccgtgaagagaagcctgctgtcaccgcagcccccaagaaatagatgccctttcttgaattgcattttttaaaacaagaaagtttccccaccagtgaatgaaagtcttgtgactagtgctgaagcttattaatgctaagggcaggcccaaattatcaagctaataaaatatcattcagcaac

SEQ ID NO: 6 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 7 - DNA, 921 bp, Homo sapiens
atagcacctgcccgcctgcctgtcctccagcaggaagtagcaagtccccagacttttgcaggcgggaaccatgtctcaggctgcaaaggcctcggcctcggccacagtggcggtgaacccagggcctgacaccaaggggaagggggccccacctgcaggaacatcccctagtcccggcactaccctggccccaacaaccgtgcctattaccagcgccaaggcggcggaactgcctcctgggaactacaggctggtggtcttcgaactggaaaacttccagggccgtcgagcagaattctcgggggagtgctcaaatctggcagaccgtggcttcgaccgtgtgcgcagcatcattgtctccgcgggaccctgggtcgcctttgagcagtccaacttccgcggggagatgttcatcctggagaagggcgagtaccctcgctggaacacatggtcgagcagctaccgcagtgatcggctcatgtccttccggcccatcaaaatggatgcccaggagcacaaaatctccctgtttgaaggggccaacttcaagggcaacaccatagagatccagggggacgacgcacccagtctctgggtctacggcttcagtgaccgcgtgggcagcgtgaaggtctccagtggaacatgggttggctatcagtatcctggctaccgcgggtaccagtacctcctagagcctggtgacttccggcactggaatgagtggggagccttccagccacagatgcagtccctgcgtcgcctgcgtgacaagcagtggcacctcgaggggtccttccctgtcctggccacagagccccccaagtgagtccacacctcactctgctaccttgccccaacccttcttcatgttcaattatttcccccatttttcccaggcaaataaaattccctaaagcc

SEQ ID NO: 8 - protein, 252 aa, Homo sapiens
MSQAAKASASATVAVNPGPDTKGKGAPPAGTSPSPGTTLAPTTVPITSAKAAELPPGNYRLVVFELENFQGRRAEFSGECSNLADRGFDRVRSIIVSAGPWVAFEQSNFRGEMFILEKGEYPRWNTWSSSYRSDRLMSFRPIKMDAQEHKISLFEGANFKGNTIEIQGDDAPSLWVYGFSDRVGSVKVSSGTWVGYQYPGYRGYQYLLEPGDFRHWNEWGAFQPQMQSLRRLRDKQWHLEGSFPVLATEPPK

SEQ ID NO: 9 - DNA, 781 bp, Homo sapiens
ctcgcaggggctggtgtcactggtcattcctgcacaggacagtccaccatggcctcagatcaccagacccaggcgggcaagccacagtccctcaaccccaagatcatcatctttgagcaggaaaactttcaaggccactcgcatgagctcaatgggccctgccccaacctgaaggaaactggcgtggagaaggcaggttctgtcctagtgcaggctggaccctgggtgggctatgaacaggccaactgcaagggcgagcagtttgtgtttgagaagggtgagtacccccgctgggactcatggaccagcagccgaaggacggactccctcagctccctgaggcccatcaaagtggacagccaagagcacaagatcatcctctatgaaaaccccaacttcaccgggaagaagatggaaatcatagatgacgatgtacccagcttccacgcccatggctaccaggagaaggtgtcatctgtgcgggtgcagagtggcacgtgggttggctaccagtaccccggctaccgtgggctgcagtacctgctggagaagggagactacaaggacagcagcgactttggggcccctcacccccaggtgcagtccgtgcgccgtatccgcgacatgcagtggcaccaacgtggtgccttccacccctccaactagtgccctccccaccatgcctccttcccaggacccaggtctgctgcccaggaaccctccagacctcccagagagtgaataaagtgtgacttgcaacttgaaaaaaaaaaaaaaaaaa

SEQ ID NO: 10 - protein, 205 aa, Homo sapiens
MASDHQTQAGKPQSLNPKIIIFEQENFQGHSHELNGPCPNLKETGVEKAGSVLVQAGPWVGYEQANCKGEQFVFEKGEYPRWDSWTSSRRTDSLSSLRPIKVDSQEHKIILYENPNFTGKKMEIIDDDVPSFHAHGYQEKVSSVRVQSGTWVGYQYPGYRGLQYLLEKGDYKDSSDFGAPHPQVQSVRRIRDMQWHQRGAFHPSN

SEQ ID NO: 11 - DNA, 843 bp, Homo sapiens
agccattcctgaatttctttcagcactgggaaaaccagtctatgcaccaaaaatgtctaaaactggaaccaagattactttctatgaagacaaaaattttcaaggccgtcgctatgactgtgattgcgactgtgcagatttccacacatacctaagtcgctgcaactccattaaagtggaaggaggcacctgggctgtttatgaaaggcccaactttgctgggtacatgtacatcttaccacagggagagtaccctgaataccagcgttggatgggcctcaacgaccgcctcagctcctgcagagctgttcatctgcctagtggaggccagtataagattcagatctttgagaaaggggattttagtggtcagatgtatgaaaccaccgaagattgcccttccatcatggagcaatttcacatgcgagagatccactcctgtaaggtgctggagggtgtctggattttctatgagctacccaactaccgtggcaggcagtacctcctggacaagaaggagtaccggaagcccatcgattggggtgcagcctccccagctgtccagtctttccgccgcattgtggagtaatgacatgaatggggccatattcttcctggggcccaaatgctggctggccttgtggtccaaataggcatcatcaataaaacagttggcatgcatcccactgctgactataatgcctctccttaaatgcttctagggaccagcaatacagtgctcgccacagtgggcagtcacacaaagctacccatctgccagatcaccaatctagatctttgtgccaaacaaaatgagatctcattaaaaggttagaaagtcaa

SEQ ID NO: 12 - protein, 178 aa, Homo sapiens
MSKTGTKITFYEDKNFQGRRYDCDCDCADFHTYLSRCNSIKVEGGTWAVYERPNFAGYMYILPQGEYPEYQRWMGLNDRLSSCRAVHLPSGGQYKIQIFEKGDFSGQMYETTEDCPSIMEQFHMREIHSCKVLEGVWIFYELPNYRGRQYLLDKKEYRKPIDWGAASPAVQSFRRIVE

SEQ ID NO: 13 - DNA, 828 bp, Homo sapiens
acatgttccctgggcctatctcggaaggggccacaatgaccctgcaatgcacaaagtcagcgggaccctggaagatggtggtgtgggatgaggacggcttccagggccggcggcacgagttcacggccgagtgccccagcgtgctggagcttggcttcgagactgtgcgatctttgaaagtgctgagtggagcgtgggtgggctttgagcatgctggcttccaagggcagcagtacattctggaacgaggcgaatatccaagctgggatgcctggggcggcaacacggcctaccccgccgagaggctcacctccttccggcctgcggcctgtgctaaccaccgtgactcgaggctgacaatcttcgagcaagagaacttcctgggcaagaaaggagagctgagcgatgactatccttccctccaggccatgggatgggaaggcaatgaagtagggtccttccacgtccactctggggcctgggtttgctcccagtttccgggctaccgaggatttcagtatgtgctggaatgcgatcaccattccggtgactacaaacatttccgggagtggggctctcatgccccgaccttccaggtgcagagcatccgcaggatccagcagtgaacaggggtgcggcacggaggagcgcatgcgtgcttatctgcaatggaggcgctctggaggctgtggtgtgttctctccttctgcctccccctgtaacctgtgtgaacccagcacccatgtgaactggtccgtgcacagtcagcacaaaaaactcaaacgaataaaaaagagaaagtctggtattaaaaaaaaaaaaaaaaaa

SEQ ID NO: 14 - protein, 196 aa, Homo sapiens
MTLQCTKSAGPWKMVVWDEDGFQGRRHEFTAECPSVLELGFETVRSLKVLSGAWVGFEHAGFQGQQYILERGEYPSWDAWGGNTAYPAERLTSFRPAACANHRDSRLTIFEQENFLGKKGELSDDYPSLQAMGWEGNEVGSFHVHSGAWVCSQFPGYRGFQYVLECDHHSGDYKHFREWGSHAPTFQVQSIRRIQQ

SEQ ID NO: 15 - DNA, 3648 bp, Homo sapiens
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 16 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 17 - DNA, 1819 bp, Homo sapiens
ggggcgacggtggtggcggcggcggcagcgggttcggttgcgcgtggcgcacggggtgggagcggagcccaggccgggagcaggcgccgccgccagtgagaaccggggccggagccgggtgcggatttgctggggctgagtcgggggcgcgcgggccctgacctctgccctctgacctctcccctagcaggcgaccatggggaacgtgttggctgccagctcgccgcccgcagggccgccaccgccgcctgcgccggccctcgtggggctgccgccacctccgccctcgccgccgggcttcacgctgccgccgctgggaggcagcctgggcgccggcaccagtacgagtcgaagttcggaacggacccccggggctgcaaccgccagcgcctcaggggccgccgaggatggggcctgcggctgcctgcccaacccgggcacattcgaggagtgccaccggaagtgcaaggagctgtttcccattcagatggagggtgtcaagctcacagtcaacaaagggttgagtaaccattttcaggtcaaccacacagtagccctcagcacaatcggggagtccaactaccacttcggggtcacatatgtggggacaaagcagctgagtcccacagaggcgttccctgtactggtgggtgacatggacaacagtggcagtctcaacgctcaggtcattcaccagctgggccccggtctcaggtccaagatggccatccagacccagcagtcgaagtttgtgaactggcaggtggacggggagtatcggggctctgacttcacagcagccgtcaccctggggaacccagacgtcctcgtgggttcaggaatcctcgtagcccactacctccagagcatcacgccttgcctggccctgggtggagagctggtctaccaccggcggcctggagaggagggcactgtcatgtctctagctgggaaatacacattgaacaactggttggcaacggtaacgttgggccaggcgggcatgcacgcaacatactaccacaaagccagtgaccagctgcaggtgggtgtggagtttgaggccagcacaaggatgcaggacaccagcgtctccttcgggtaccagctggacctgcccaaggccaacctcctcttcaaaggctctgtggatagcaactggatcgtgggtgccacgctggagaagaagctcccacccctgcccctgacactggcccttggggccttcctgaatcaccgcaagaacaagtttcagtgtggctttggcctcaccatcggctgagccctcctggcccccgccttccacgcccttccgattccacctccacctccacctccccctgccacagaggggagacctgagcccccctcccttccctccccccttgggggtcgggggggacattggaaaggagggaccccgccaccccagcagctgaggaggggattctggaactgaatggcgcttcgggattctgagtagcaggggcagcatgcccagtgggcctggggtcccgggagggattccggaattgaggggcacgcaggattctgagcaccaggggcagaggcggccagacaacctcagggaggagtgtcctggcgtccccatcctccaaagggcctgggcccgccccgagggggcagcgagaggagcttccccatccccggtcagtccaccctgccccgtccactttcccatctcctcggtataaatcatgtttataagttatggaagaaccgggacattttacagaaaaaaaacaaaaaacaacaaaaaatatacgtgggaaaaaaaacgaaaaaaaaaaaaaaaaaa

SEQ ID NO: 18 - protein, 361 aa, Homo sapiens
MGNVLAASSPPAGPPPPPAPALVGLPPPPPSPPGFTLPPLGGSLGAGTSTSRSSERTPGAATASASGAAEDGACGCLPNPGTFEECHRKCKELFPIQMEGVKLTVNKGLSNHFQVNHTVALSTIGESNYHFGVTYVGTKQLSPTEAFPVLVGDMDNSGSLNAQVIHQLGPGLRSKMAIQTQQSKFVNWQVDGEYRGSDFTAAVTLGNPDVLVGSGILVAHYLQSITPCLALGGELVYHRRPGEEGTVMSLAGKYTLNNWLATVTLGQAGMHATYYHKASDQLQVGVEFEASTRMQDTSVSFGYQLDLPKANLLFKGSVDSNWIVGATLEKKLPPLPLTLALGAFLNHRKNKFQCGFGLTIG

SEQ ID NO: 19 - DNA, 2877 bp, Homo sapiens
gctttccgcggcattctttgggcgtgagtcatgcaggtttgcagccagccccaaagggggtgtgtgcgcgagcagagcgctataaatacggcgcctcccagtgcccacaacgcggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcacactgtttcaaaagttaaaaaaaaaaaaaaaaaa

SEQ ID NO: 20 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 21 - DNA, 5657 bp, Homo sapiens
acaagtctttccgcctccccagcccgcccgggagctgcgagccgcgagctggattatggtggcctgagcagccaacgcagccgcaggagcccggagcccttgcccctgcccgcgccgccgcccgccggggggaccagggaagccgccaccggcccgccatgcccgcccctcccagccccgccgggagcccgcgcccgctgcccaggctggccgccgccgtgccgatgtagcgggctccggatcccagcctctcccctgctcccgtgctctgcggatctcccctgaccgctctccacagcccggacccgggggctggcccagggccctgcaggccctggcgtcctgatgcccccaagctccctctcctgagaagccaccagcaccacccagacttgggggcaggcgccagggacggacgtgggccagtgcgagcccagagggcccgaaggccggggcccaccatggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctgctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtgaggaggcccatgggcagaagatagagattcccctggaccacacctccgtggttcactttggtcacaagtaggagacacagatggcacctgtggccagagcacctcaggaccctccccacccaccaaatgcctctgccttgatggagaaggaaaaggctggcaaggtgggttccagggactgtacctgtaggaaacagaaaagagaagaaagaagcactctgctggcgggaatactcttggtcacctcaaatttaagtcgggaaattctgctgcttgaaacttcagccctgaacctttgtccaccattcctttaaattctccaacccaaagtattcttcttttcttagtttcagaagtactggcatcacacgcaggttaccttggcgtgtgtccctgtggtaccctggcagagaagagaccaagcttgtttccctgctggccaaagtcagtaggagaggatgcacagtttgctatttgctttagagacagggactgtataaacaagcctaacattggtgcaaagattgcctcttgaattaaaaaaaaaaactagattgactatttatacaaatgggggcggctggaaagaggagaaggagagggagtacaaagacagggaatagtgggatcaaagctaggaaaggcagaaacacaaccactcaccagtcctagttttagacctcatctccaagatagcatcccatctcagaagatgggtgttgttttcaatgttttcttttctgtggttgcagcctgaccaaaagtgagatgggaagggcttatctagccaaagagctcttttttagctctcttaaatgaagtgcccactaagaagttccacttaacacatgaatttctgccatattaatttcattgtctctatctgaaccaccctttattctacatatgataggcagcactgaaatatcctaaccccctaagctccaggtgccctgtgggagagcaactggactatagcagggctgggctctgtcttcctggtcataggctcactctttcccccaaatcttcctctggagctttgcagccaaggtgctaaaaggaataggtaggagacctcttctatctaatccttaaaagcataatgttgaacattcattcaacagctgatgccctataacccctgcctggatttcttcctattaggctataagaagtagcaagatctttacataattcagagtggtttcattgccttcctaccctctctaatggcccctccatttatttgactaaagcatcacacagtggcactagcattataccaagagtatgagaaatacagtgctttatggctctaacattactgccttcagtatcaaggctgcctggagaaaggatggcagcctcagggcttccttatgtcctccaccacaagagctccttgatgaaggtcatctttttcccctatcctgttcttcccctccccgctcctaatggtacgtgggtacccaggctggttcttgggctaggtagtggggaccaagttcattacctccctatcagttctagcatagtaaactacggtaccagtgttagtgggaagagctgggttttcctagtatacccactgcatcctactcctacctggtcaacccgctgcttccaggtatgggacctgctaagtgtggaattacctgataagggagagggaaatacaaggagggcctctggtgttcctggcctcagccagctgcccacaagccataaaccaataaaacaagaatactgagtcagttttttatctgggttctcttcattcccactgcacttggtgctgctttggctgactgggaacaccccataactacagagtctgacaggaagactggagactgtccacttctagctcggaacttactgtgtaaataaactttcagaactgctaccatgaagtgaaaatgccacattttgctttataatttctacccatgttgggaaaaactggctttttcccagccctttccagggcataaaactcaaccccttcgatagcaagtcccatcagcctattatttttttaaagaaaacttgcacttgtttttctttttacagttacttccttcctgccccaaaattataaactctaagtgtaaaaaaaagtcttaacaacagcttcttgcttgtaaaaatatgtattatacatctgtatttttaaattctgctcctgaaaaatgactgtcccattctccactcactgcatttggggcctttcccattggtctgcatgtcttttatcattgcaggccagtggacagagggagaagggagaacaggggtcgccaacacttgtgttgctttctgactgatcctgaacaagaaagagtaacactgaggcgctcgctcccatgcacaactctccaaaacacttatcctcctgcaagagtgggctttccagggtctttactgggaagcagttaagccccctcctcaccccttccttttttctttctttactcctttggcttcaaaggattttggaaaagaaacaatatgctttacactcattttcaatttctaaatttgcaggggatactgaaaaatacggcaggtggcctaaggctgctgtaaagttgaggggagaggaaatcttaagattacaagataaaaaacgaatcccctaaacaaaaagaacaatagaactggtcttccattttgccacctttcctgttcatgacagctactaacctggagacagtaacatttcattaaccaaagaaagtgggtcacctgacctctgaagagctgagtactcaggccactccaatcaccctacaagatgccaaggaggtcccaggaagtccagctccttaaactgacgctagtcaataaacctgggcaagtgaggcaagagaaatgaggaagaatccatctgtgaggtgacaggcaaggatgaaagacaaagaaggaaaagagtatcaaaggcagaaaggagatcatttagttgggtctgaaaggaaaagtctttgctatccgacatgtactgctagtacctgtaagcattttaggtcccagaatggaaaaaaaaatcagctattggtaatataataatgtcctttccctggagtcagtttttttaaaaagttaactcttagtttttacttgtttaattctaaaagagaagggagctgaggccattccctgtaggagtaaagataaaaggataggaaaagattcaaagctctaatagagtcacagctttcccaggtataaaacctaaaattaagaagtacaataagcagaggtggaaaatgatctagttcctgatagctacccacagagcaagtgatttataaatttgaaatccaaactactttcttaatatcactttggtctccatttttcccaggacaggaaatatgtccccccctaactttcttgcttcaaaaattaaaatccagcatcccaagatcattctacaagtaattttgcacagacatctcctcaccccagtgcctgtctggagctcacccaaggtcaccaaacaacttggttgtgaaccaactgccttaaccttctgggggagggggattagctagactaggagaccagaagtgaatgggaaagggtgaggacttcacaatgttggcctgtcagagcttgattagaagccaagacagtggcagcaaaggaagacttggcccaggaaaaacctgtgggttgtgctaatttctgtccagaaaatagggtggacagaagcttgtggggtacatggaggaattgggacctggttatgttgttattctcggactgtgaattttggtgatgtaaaacagaatattctgtaaacctaatgtctgtataaataatgagcgttaacacagtaaaatattcaataagaagtcaaactactagggttaaaaaaaaaaaaaaaaaa

SEQ ID NO: 22 - protein, 432 aa, Homo sapiens
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 23 - DNA, 6107 bp, Homo sapiens
aaatgacgacaacggtgagggttctcgggcggggcctgggacaggcagctccggggtccgcggtttcacatcggaaacaaaacagcggctggtctggaaggaacctgagctacgagccgcggcggcagcggggcggcggggaagcgtatacctaatctgggagcctgcaagtgacaacagcctttgcggtccttagacagcttggcctggaggagaacacatgaaagaaagaacctcaagaggctttgttttctgtgaaacagtatttctatacagttgctccaatgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctagcatatttgcggttagaatcccatggatgtttcttctttgactataacaaaatctggggaggacaaaggtgattttcctgtgtccacatctaacaaagtcaagattcccggctggacttttgcagcttccttccaagtcttcctgaccaccttgcactattggactttggaaggaggtgcctatagaaaacgattttgaacatacttcatcgcagtggactgtgtccctcggtgcagaaactaccagatttgagggacgaggtcaaggagatatgataggcccggaagttgctgtgccccatcagcagcttgacgcgtggtcacaggacgatttcactgacactgcgaactctcaggactaccgttaccaagaggttaggtgaagtggtttaaaccaaacggaactcttcatcttaaactacacgttgaaaatcaacccaataattctgtattaactgaattctgaacttttcaggaggtactgtgaggaagagcaggcaccagcagcagaatggggaatggagaggtgggcaggggttccagcttccctttgattttttgctgcagactcatcctttttaaatgagacttgttttcccctctctttgagtcaagtcaaatatgtagattgcctttggcaattcttcttctcaagcactgacactcattaccgtctgtgattgccatttcttcccaaggccagtctgaacctgaggttgctttatcctaaaagttttaacctcaggttccaaattcagtaaattttggaaacagtacagctatttctcatcaattctctatcatgttgaagtcaaatttggattttccaccaaattctgaatttgtagacatacttgtacgctcacttgccccagatgcctcctctgtcctcattcttctctcccacacaagcagtctttttctacagccagtaaggcagctctgtcgtggtagcagatggtcccattattctagggtcttactctttgtatgatgaaaagaatgtgttatgaatcggtgctgtcagccctgctgtcagaccttcttccacagcaaatgagatgtatgcccaaagacggtagaattaaagaagagtaaaatggctgttgaagcactttctgtcctggtattttgtttttgcttttgccacacagtagctcagaatttgaacaaatagccaaaagctggtggttgatgaattatgaactagttgtatcaacacaaagcaagagttggggaaagccatatttaacttggtgagctgtgggagaacctggtggcagaaggagaaccaactgccaaggggaaagagaaggggcctccagcagcgaaggggatacagtgagctaatgatgtcaaggaggagtttcaggttattctcgtcagctccacaaatgggtgctttgtggtctctgcccgcgttacctttcctctcaatgtacctttgtgtgaactgggcagtggaggtgcctgctgcagttaccatggagttcaggctctgggcagctcagtcaggcaaaacacacaaacagccatcagcctgtgtgggctcagggcacctctggacaaaggcttgtggggcataaccttctttaccacagagagcccttagctatgctgatcagaccgtaagcgtttatgagaaacttagtttcctcctgtggctgaggaggggccagctttttcttcttttgcctgctgttttctctcccaatctatgatatgatatgacctggtttggggctgtctttggtgtttagaatatttgttttctgtcccaggatatttcttataagaacctaacttcaagagtagtgtgcgagtactgatctgaatttaaattaaaattggcttatattaggcagtcacagacaggaaaaataagagctatgcaaagaaagggggatttaaagtagtaggttctatcatctcaattcatttttttccatgaaatcccttcttccaagattcattccctctctcagacatgtgctagcatgggtattatcattgagaaagcacagctacagcaaagccacctgaatagcaatttgtgattggaagcattcttgagggatccctaatctagagtaatttatttgtgtaaggatcccaaatgtgttgcacctttcatgatacatttcttctctgaagagggtacgtggggtgtgtgtatttaaatccatcctatgtattactgattgtcctgtgtagaaagatggcaattattctgtctctttctccaagtttgagccacatctcagccacattgttagacagtgtacagagaacctatctttcctttttttttttttaaaggacaggattttgctgtgttgcccaggctagacttgaactcctgggctcaagtaatccacctcagcctgagtagctgagactacagcccatcttatttctttaaatcattcatctcaggcagagaacttttccctcaaacattctttttagaattagttcagtcattcctaaaacatccaaatgctagtcttccaccatgaaaaatagattgtcactggaaagaacagtagcaatttccataaggatgtgccttcactcacacgggacaggcggtggttatagagtcgggcaaaaccagcagtagagtatgaccagccaagccaatctgcttaataaaaagatggaagacagtaaggaaggaaagtagccactaagagtctgagtctgactgggctacagaataaagggtatttatggacagaatgtcattacatgcctatgggaataccaatcatatttggaagatttgcagattttttttcagagaggaaagactcaccttcctgtttttggttctcagtaggttcgtgtgtgttcctagaatcacagctctgactccaaatgactcaatttctcaattagaaaaagtagaagctttctaagcaacttggaagaaaacagtcataagtaagcaatttgttgattttactacagaagcaacaactgaagaggcagtgtttttactttcagactccgggattcccattctgtagtctctctgcttttaaaaaccctccttttgcaatagatgcccaaacagatgatgtttattacttgttatttacgtggcctcagacagtgtatgtattctcgatataacttgtagagtgtgaaatataagtttaactaccaaataaggtctcccagggttagatgactgcgggaagcctttgatcccaacccccaaggctttgtatatttgatcatttgtgatctaaccctggaagaaaaagagctcagaaaccactatgaaaaaatttgttcagtgttttctgtgttcccgtaggttctggagtctgaggatgcaaagatgaataagataaattctcagaatgtagttataatctcttgttttctggtatatgccatctttctttaacttctctaaaatattgggtatttgtcaaataaccacttttaacagttaccattactgagggcttatacattggtgttataaaagtgacttgattcagaaatcaatccattcagtaaagtactccttctctaaatttgctgttatgtctataaggaacagtttgacctgcccttctcctcacctcctcacctgccttccaacattgaatttggaaggagacgtgaaaattggacatttggttttgcccttgggctggaaactatcatataatcataagtttgagcctagaagtgatccttgtgatcttctcacctctttaaattcccacaacacaagagattaaaaacagaggtttcagctcttcatagtgcgttgtgaaatggctggccagagtgtaccaacaaagctgtcatcgggctcacagctcagagacatctgcatgtgatcatctgcatagtcctctcctctaacgggaaacacctcagatttgcatataaaaaagcaccctggtgctgaaatgaacccctttcttgaacatcaaagctgtctcccacagccttgggcagcagggtgcctcttagtggatgtgctgggtccaccctgagccctgacatgtggtggcagcattgccagttggtctgtgtgtctgtgtagcagggacgatttcccagaaagcaattttccttttgaaatacgtaattgttgagactaggcagtttcaaagtcagctgcatatagtagcaagtacaggactgtcttgtttttggtgtccttggaggtgctggggtgagggtttcagtgggatcatttactctcacatgttgtctgccttctgcttctgtggacactgctttgtacttaattcagacagactgtgaatacaccttttttataaatacctttcaaattcttggtaagatataattttgatagctgattgcagattttctgtatttgtcagattaataaagactgcatgaatccaaaaaaaaaaaaaaaaaa

SEQ ID NO: 24 - protein, 467 aa, Homo sapiens
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 25 - DNA, 3677 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 26 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 27 - DNA, 7123 bp, Homo sapiens
atcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacgagaggacggactctggcggccgggtcgttggccgcggggagcgcgggcaccgggcgagcaggccgcgtcgcgctcaccatggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatacgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctagagtttgacacgaagccttatttctagaagcacatgtgtatttatacccccaggaaactagcttttgccagtattatgcatatataagtttacacctttatctttccatgggagccagctgctttttgtgatttttttaatagtgcttttttttttttgactaacaagaatgtaactccagatagagaaatagtgacaagtgaagaacactactgctaaatcctcatgttactcagtgttagagaaatccttcctaaacccaatgacttccctgctccaacccccgccacctcagggcacgcaggaccagtttgattgaggagctgcactgatcacccaatgcatcacgtaccccactgggccagccctgcagcccaaaacccagggcaacaagcccgttagccccagggatcactggctggcctgagcaacatctcgggagtcctctagcaggcctaagacatgtgaggaggaaaaggaaaaaaagcaaaaagcaagggagaaaagagaaaccgggagaaggcatgagaaagaatttgagacgcaccatgtgggcacggagggggacggggctcagcaatgccatttcagtggcttcccagctctgacccttctacatttgagggcccagccaggagcagatggacagcgatgaggggacattttctggattctgggaggcaagaaaaggacaaatatcttttttggaactaaagcaaattttagaactttacctatggaagtggttctatgtccattctcattcgtggcatgttttgatttgtagcactgagggtggcactcaactctgagcccatacttttggctcctctagtaagatgcactgaaaacttagccagagttaggttgtctccaggccatgatggccttacactgaaaatgtcacattctattttgggtattaatatatagtccagacacttaactcaatttcttggtattattctgttttgcacagttagttgtgaaagaaagctgagaagaatgaaaatgcagtcctgaggagaggagttttctccatatcaaaacgagggctgatggaggaaaaaggtcaataaggtcaagggaaaaccccgtctctataccaaccaaaccaattcaccaacacagttgggacccaaaacacaggaagtcagtcacgtttccttttcatttaatggggattccactatctcacactaatctgaaaggatgtggaagagcattagctggcgcatattaagcactttaagctccttgagtaaaaaggtggtatgtaatttatgcaaggtatttctccagttgggactcaggatattagttaatgagccatcactagaagaaaagcccattttcaactgctttgaaacttgcctggggtctgagcatgatgggaatagggagacagggtaggaaagggcgcctactcttcagggtctaaagatcaagtgggccttggatcgctaagctggctctgtttgatgctatttatgcaagttagggtctatgtatttatgatgtctgcaccttctgcagccagtcagaagctggagaggcaacagtggattgctgcttcttggggagaagagtatgcttccttttatccatgtaatttaactgtagaacctgagctctaagtaaccgaagaatgtatgcctctgttcttatgtgccacatccttgtttaaaggctctctgtatgaagagatgggaccgtcatcagcacattccctagtgagcctactggctcctggcagcggcttttgtggaagactcactagccagaagagaggagtgggacagtcctctccaccaagatctaaatccaaacaaaagcaggctagagccagaagagaggacaaatctttgttcttcctcttctttacatacgcaaaccacctgtgacagctggcaattttataaatcaggtaactggaaggaggttaaacacagaaaaaagaagacctcagtcaattctctacttttttttttttttccaaatcagataatagcccagcaaatagtgataacaaataaaaccttagctattcatgtcttgatttcaataattaattcttaatcattaagagaccataataaatactccttttcaagagaaaagcaaaaccattagaattgttactcagctccttcaaactcaggtttgtagcatacatgagtccatccatcagtcaaagaatggttccatctggagtcttaatgtagaaagaaaaatggagacttgtaataatgagctagttacaaagtgcttgttcattaaaatagcactgaaaattgaaacatgaattaactgataatattccaatcatttgccatttatgacaaaaatggttggcactaacaaagaacgagcacttcctttcagagtttctgagataatgtacgtggaacagtctgggtggaatggggctgaaaccatgtgcaagtctgtgtcttgtcagtccaagaagtgacaccgagatgttaattttagggacccgtgccttgtttcctagcccacaagaatgcaaacatcaaacagatactcgctagcctcatttaaattgattaaaggaggagtgcatctttggccgacagtggtgtaactgtatgtgtgtgtgtgtgtgtgtgtgtgtgtgtgtgtgtgggtgtatgtgtgttttgtgcataactatttaaggaaactggaattttaaagttacttttatacaaaccaagaatatatgctacagatataagacagacatggtttggtcctatatttctagtcatgatgaatgtattttgtataccatcttcatataataaacttccaaaaacaca

SEQ ID NO: 28 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLFIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 29 - DNA, 6055 bp, Homo sapiens
actgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacacccccaactcctggacatcacatgagaggtgctgctcagattttcaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctctttttcattcatttaaaaagcatttatcatgccccctgctgcgggtctcaccatgggtttagaacaaagacgttcaagaaatggccccatcctcaaagaagtagcagtacctggggagctgacacttctgtaaaactagaagataaaccaggcaatgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttacgtgtctcctggccaagccccaggaaggaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttgaaggaatccctttgcaaggttgcattactgtacccatcatttctaaaatggaagagggggtggctgggcacagtggccgacacctaaaaacccagcactttggggggccaaggtgggaggatcgcttgagcccaggagttcaagaccagtctggccaacatggtcagattccatctcaaagaaaaaaggtaaaaataaaataaaatggagaagaaggaatcaga

SEQ ID NO: 30 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 31 - DNA, 1552 bp, Homo sapiens
ggtcgctttaagaaaggagtagctgtaatctgaagcctgctggacgctggattagaaggcagcaaaaaaagctctgtgctggctggagccccctcagtgtgcaggcttagagggactaggctgggtgtggagctgcagcgtatccacaggccccaggatgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaatatcccagtttaatattccaataccctagaagaaaacccgagggacagcagattccacaggacacgaaggctgcccctgtaaggtttcaatgcatacaataaaagagctttatccctaaaaaaaaaaaaaaaaaaaaa

SEQ ID NO: 32 - protein, 418 aa, Homo sapiens
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 33 - DNA, 2693 bp, Homo sapiens
gcttgttccctgtcctctggccctttgcaaataaatgccttaccagacctgccctgccaccccactcgcagccacccagcaagagcagcatgtcagcctgccggagctttgcagttgcaatctgcattttagaaataagcatcctcacagcacagtacacgaccagttatgacccagagctaacagaaagcagtggctctgcatcacacatagactgcagaatgagcccctggagtgaatggtcacaatgcgatccttgtctcagacaaatgtttcgttcaagaagcattgaggtctttggacaatttaatgggaaaagatgcaccgacgctgtgggagacagacgacagtgtgtgcccacagagccctgtgaggatgctgaggatgactgcggaaatgactttcaatgcagtacaggcagatgcataaagatgcgacttcggtgtaatggtgacaatgactgcggagacttttcagatgaggatgattgtgaaagtgagccccgtcccccctgcagagacagagtggtagaagagtctgagctggcacgaacagcaggctatgggatcaacattttagggatggatcccctaagcacaccttttgacaatgagttctacaatggactctgtaaccgggatcgggatggaaacactctgacatactaccgaagaccttggaacgtggcttctttgatctatgaaaccaaaggcgagaaaaatttcagaaccgaacattacgaagaacaaattgaagcatttaaaagtatcatccaagagaagacatcaaattttaatgcagctatatctctaaaatttacacccactgaaacaaataaagctgaacaatgttgtgaggaaacagcctcctcaatttctttacatggcaagggtagttttcggttttcatattccaaaaatgaaacttaccaactatttttgtcatattcttcaaagaaggaaaaaatgtttctgcatgtgaaaggagaaattcatctgggaagatttgtaatgagaaatcgcgatgttgtgctcacaacaacttttgtggatgatataaaagctttgccaactacctatgaaaagggagaatattttgcctttttggaaacctatggaactcactacagtagctctgggtctctaggaggactctatgaactaatatatgttttggataaagcttccatgaagcggaaaggtgttgaactaaaagacataaagagatgccttgggtatcatctggatgtatctctggctttctctgaaatctctgttggagctgaatttaataaagatgattgtgtaaagaggggagagggtagagctgtaaacatcaccagtgaaaacctcatagatgatgttgtttcactcataagaggtggaaccagaaaatatgcatttgaactgaaagaaaagcttctccgaggaaccgtgattgatgtgactgactttgtcaactgggcctcttccataaatgatgctcctgttctcattagtcaaaaactgtctcctatatataatctggttccagtgaaaatgaaaaatgcacacctaaagaaacaaaacttggaaagagccattgaagactatatcaatgaatttagtgtaagaaaatgccacacatgccaaaatggaggtacagtgattctaatggatggaaagtgtttgtgtgcctgcccattcaaatttgagggaattgcctgtgaaatcagtaaacaaaaaatttctgaaggattgccagccctagagttccccaatgaaaaatagagctgttggcttctctgagctccagtggaagaagaaaacactagtaccttcagatcctacccctgaagataatcttagctgccaagtaaatagcaacatgcttcatgaaaatcctaccaacctctgaagtctcttctctcttaggtctataatttttttttaaatttttcttccttaaactcctgtgatgtttccattttttgttccctaatgagaagtcaacagtgaaatacgccagaactgctttatcccacggaaaatgccaatctcttctaaaaaaaaacaaaattaaattaaaaacagaatgttggtttaaaaaacttcaaagtaattttcaaacggctttgtatggttaacatattctgccaggtccatgaccacacgtctgtaccatgcaatttaactcttatttacattgttatgtttagtttggttatttgcttaggtgtgcatacattcattcagcaaatgctgagcaccagccacgtgcacagcagttgcttttactagtcttagctctacgatttaaatccatgtgtccaagggggaaaacatattatatttgtaaccaaaaactactagtttaccagaggactgaagggagataaagaggagttggttaatgggtacaaaaatccagttagatgaaaggaataatatagatagtgttcagtagcagaatagaatgaacataaactattagtttaaattatgtgaaattccttctatttgatcatattttacaagaaaaaacatcaattttatatagtccaacttaatacctagccttatgagttgtataaggtaaggttacctacctgagaagctgattaacattggttgtacaatcttattcattagagaacatggtgcttagggtctgagaccttttgaaaggtctgagaactctttaaaaaaaggaaa

SEQ ID NO: 34 - protein, 559 aa, Homo sapiens
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 35 - DNA, 960 bp, Homo sapiens
gggcatgaatatcagacgctagggggacagccactgtgttgtctgctaccctcatcctggtcactgcttctgctataacagccctaggccaggaatatgaacaagccgctgctttggatctctgtcctcaccagcctcctggaagcctttgctcacacagacctcagtgggaaggtgtttgtatttcctagagaatctgttactgatcatgtaaacttgatcacaccgctggagaagcctctacagaactttaccttgtgttttcgagcctatagtgatctctctcgtgcctacagcctcttctcctacaatacccaaggcagggataatgagctactagtttataaagaaagagttggagagtatagtctatacattggaagacacaaagttacatccaaagttatcgaaaagttcccggctccagtgcacatctgtgtgagctgggagtcctcatcaggtattgctgaattttggatcaatgggacacctttggtgaaaaagggtctgcgacagggttactttgtagaagctcagcccaagattgtcctggggcaggaacaggattcctatgggggcaagtttgataggagccagtcctttgtgggagagattggggatttgtacatgtgggactctgtgctgcccccagaaaatatcctgtctgcctatcagggtacccctctccctgccaatatcctggactggcaggctctgaactatgaaatcagaggatatgtcatcatcaaacccttggtgtgggtctgaggtcttgactcaacgagagcacttgaaaatgaaatgactgtctaagagatctggtcaaagcaactggatactagatcttacatctgcagctctttcttctttgaatttcctatctgtatgtctgcctaattaaaaaaatatatattgtattatgctacctgcaaaaaaaaaaaaaaaaaaaaaaaaaaaaaa

SEQ ID NO: 36 - protein, 223 aa, Homo sapiens
MNKPLLWISVLTSLLEAFAHTDLSGKVFVFPRESVTDHVNLITPLEKPLQNFTLCFRAYSDLSRAYSLFSYNTQGRDNELLVYKERVGEYSLYIGRHKVTSKVIEKFPAPVHICVSWESSSGIAEFWINGTPLVKKGLRQGYFVEAQPKIVLGQEQDSYGGKFDRSQSFVGEIGDLYMWDSVLPPENILSAYQGTPLPANILDWQALNYEIRGYVIIKPLVWV

SEQ ID NO: 37 - DNA, 5496 bp, Homo sapiens
ctgtggcttgccccagagctgatccttgtctttgtccacttctcagcgaggatggcacttcagggagcccttcccttactatcgcagagagagcaggccctccccagtcatgtccaacccagaactctgttttgttttcttcatagccctagcatcacagaaaatcaccctgtgcattcatggatgtccacgggggcaagggctttgtgttgcttaacccagcatcctgaaccgtgtttgttgaatgaatacagaaccccgtttgctctgggagagcacagaaaacagtcttctatcatatatcatagccagctgcaaacagcagatggcttcccatatcccagagagtaagaaccagagagagagagaaagagagagagtttgggtctttctcctctgtgcctgctctctccagagaaactggaggggtagcagttagcattcccccgctggttccaccaagcacagtcaaggtctctaggacatggccacccctcacctgtggaagcggtcctgctggggtgggtgggtgttagttggttctggtttgggtcagagacacccagtggcccaggtgggcgtggggccagggcgcagacgagaaggggcacgagggctccgctccgaggacccagcggcaagcaccggtcccgggcgcgccccagcccacccactcgcgtgcccacggcggcattattccctataaggatctgaacgatccgggggcggccccgccccgttaccccttgcccccggccccgccccctttttggagggccgatgaggtaatgcggctctgccattggtctgagggggcgggccccaacagcccgaggcggggtccccgggggcccagcgctatatcactcggccgcccaggcagcggcgcagagcgggcagcaggcaggcggcgggcgctcagacggcttctcctcctcctcttgctcctccagctcctgctccttcgccgggaggccgcccgccgagtcctgcgccagcgccgaggcagcctcgctgcgccccatcccgtcccgccgggcactcggagggcagcgcgccggaggccaaggttgccccgcacggcccggcgggcgagcgagctcgggctgcagcagccccgccggcggcgcgcacggcaactttggagaggcgagcagcagccccggcagcggcggcagcagcggcaatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccatacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatctctatttttttaggaaaacaaaaatgaaaaactactccatttgaggattgtaattcccacccctcttgcttcttccccacctcaccatctcccagaccctcttccctttgcccttctcctccaatacataaaggacacagacaaggaacttgctgaaaggccaaccatttcaggatcagtcaaaggcagcaagcagatagactcaaggtgtgtgaaagatgttatacaccaggagctgccactgcatgtcccaaccagactgtgtctgtctgtgtctgcatgtaagagtgagggagggaaggaaggaactacaagagagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatggggacataagctaatttttttacaggacacagaattctgttcaatgctgttaaatatgccaatagtttaatctcttctattttgttgtcgttgcttgtttgaagaaaatcatgacattccaagttgacatttttttttcattttaattaaaatttgaaattctgaacaccgtcagcaccctctcttccctatcatgggtcatctgacccctgtccgtctccttgtccctgcttcatgtttgggggcctttctttaactgccttcctggcttagctcagatggcagatgagagtgtagtcaagggcctgggcacaggagggagagctgcagagtgtcctgcctgccttggctggagggacacctctcctgggtgtggagacagcttggttccctttccctagctccctggtgggtgaatgccacctcctgagatcctcacctcttggaattaaaattgttggtcactggggaaagcctgagtttgcaaccagttgtagggtttctgttgtgtttttttttttttttttgaaataaaactataatataaattctcctattaaataaaattattttaagttttagtgtcaaaagtgagatgctgagagtaggtgataatgtatattttacagagtgggggttggcaggatggtgacattgaacatgattgctctctgtctcttttttcagcttatgggtatttatcttctattagtatttgtatcttcagttcattccactttaggaaacagagctgccaattgaaacagaagaagaaaaaaaaaaaaagcagcagacaacacactgtagagtcttgcacacacacaagtgcccaggcaaggtgcttggcagaaccgcagagtgggaagagagtaccggcatcgggtttccttgggatcaatttcattaccgtgtacctttcccattgtggtcatgccatttggcagggggagaatgggaggcttggccttctttgtgaggcagtgtgagcagaagctgatgccagcatgtcactggttttgaagggatgagcccagacttgatgttttgggattgtccttattttaacctcaaggtctcgcatggtggggcccctgaccaacctacacaagttccctcccacaagtggacatcagtgtcttctctgtgaggcatctggccattcgcactccctggtgtggtcagcctctctcacacaaggaggaacttgggtgaaggctgagtgtgaggcacctgaagtttccctgcggagtcgataaattagcagaaccacatccccatctgttaggccttggtgaggaggccctgggcaaagaagggtctttcgcaaagcgatgtcagagggcggttttgagctttctataagctatagctttgtttatttcacccgttcacttactgtataatttaaaatcatttatgtagctgagacacttctgtatttcaatcatatcatgaacattttattttgctaaatcttgtgtcatgtgtaggctgtaatatgtgtacattgtgtttaagagaaaaatgaaacccacatgccgccattttcctgaatcaaattctgcagtggaatggagaggaaaatacttctaggcaagcagctagactggtgaattgggggaaatagaaggaactagtaactgagactcctccagcctcctccctattggaatcccaatggctcctggagtaggaaaaaagtttaaactacattcatgttcttgttctgtgtcactcggccctgggtagtctaccatttacttcaccccaagtcctgctgcccatccagttgggaagccatgattttcctaagaatccagggccatgggagatacaattccaagttctcgcttcctcctttgggcatctcttctgcctcccaatcaaggaagctccatgctcaggctctcagctctcgggccagtgctctgctctgtccagggtaggtaatactgggagactcctgtcttttaccctcccctcgttccagacctgcctcatggtggcaacatggttcttgaacaattaaagaaacaaatgactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatgaattttatattccgtgaatgtatattgtcttgtaatgttgcataatgttcactttttatagtgtgtcctttattctaaacagtaaagtggttttatttctatcacaaaaaaaaaaa

SEQ ID NO: 38 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 39 - DNA, 1678 bp, Homo sapiens
gagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccatggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctgtaggagtcagagcccacatggccgggccctctgtagctccctcctcccatctccttcccccagcccaataaaggtcccttagccccgagtttaaa

SEQ ID NO: 40 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 41 - DNA, 26 bp, Artificial Sequence
  Description of Artificial Sequence Synthetic primer
ggtgtctgtcttcctttgcttccctt

SEQ ID NO: 42 - DNA, 22 bp, Artificial Sequence
  Description of Artificial Sequence Synthetic primer
taagctctcctggctgctctct

SEQ ID NO: 43 - DNA, 26 bp, Artificial Sequence
  Description of Artificial Sequence Synthetic primer
aggtgttgggagatgtgattgaggtg

SEQ ID NO: 44 - DNA, 26 bp, Artificial Sequence
  Description of Artificial Sequence Synthetic primer
acagggatgaagtaatggtgagaggg

SEQ ID NO: 45 - DNA, 20 bp, Artificial Sequence
  Description of Artificial Sequence Synthetic primer
caggaacatcccctagtccc

SEQ ID NO: 46 - DNA, 21 bp, Artificial Sequence
  Description of Artificial Sequence Synthetic primer
gtctgccagatttgacgactc

SEQ ID NO: 47 - DNA, 24 bp, Artificial Sequence
  Description of Artificial Sequence Synthetic primer
aagggagagcagtttgtgtttgag

SEQ ID NO: 48 - DNA, 24 bp, Artificial Sequence
  Description of Artificial Sequence Synthetic primer
cttgtgctcttggctgtccacttt

SEQ ID NO: 49 - DNA, 28 bp, Artificial Sequence
  Description of Artificial Sequence Synthetic primer
gtatgaaaccaccgaagattgcccttcc

SEQ ID NO: 50 - DNA, 22 bp, Artificial Sequence
  Description of Artificial Sequence Synthetic primer
agacaccctccagcaccttaca

SEQ ID NO: 51 - DNA, 26 bp, Artificial Sequence
  Description of Artificial Sequence Synthetic primer
atgggatgggaaggcaatgaagtagg

SEQ ID NO: 52 - DNA, 27 bp, Artificial Sequence
  Description of Artificial Sequence Synthetic primer
ccggaaatgtttgtagtcaccggaatg

SEQ ID NO: 53 - DNA, 24 bp, Artificial Sequence
  Description of Artificial Sequence Synthetic primer
ccgctgcttagttggtgagtttgt

SEQ ID NO: 54 - DNA, 24 bp, Artificial Sequence
  Description of Artificial Sequence Synthetic primer
acggtgtgccagtgaagatgagtt

SEQ ID NO: 55 - DNA, 20 bp, Artificial Sequence
  Description of Artificial Sequence Synthetic primer
aactggcactgggtcgcttt

SEQ ID NO: 56 - DNA, 25 bp, Artificial Sequence
  Description of Artificial Sequence Synthetic primer
gccttcaactccttcatggtctcgt

SEQ ID NO: 57 - DNA, 26 bp, Artificial Sequence
  Description of Artificial Sequence Synthetic primer
atttatggagaccgtggcggagaaag

SEQ ID NO: 58 - DNA, 24 bp, Artificial Sequence
  Description of Artificial Sequence Synthetic primer
ctggttacttggtgacgtgcagag

SEQ ID NO: 59 - DNA, 29 bp, Artificial Sequence
  Description of Artificial Sequence Synthetic primer
cagacaagttcttcatcaacggctccaac

SEQ ID NO: 60 - DNA, 23 bp, Artificial Sequence
  Description of Artificial Sequence Synthetic primer
ttgggaacgtgggtctgctttac

SEQ ID NO: 61 - DNA, 26 bp, Artificial Sequence
  Description of Artificial Sequence Synthetic primer
gagttacctgcaccgttgtcctactt

SEQ ID NO: 62 - DNA, 24 bp, Artificial Sequence
  Description of Artificial Sequence Synthetic primer
tgtgctcctgccgttctctattgt

SEQ ID NO: 63 - DNA, 20 bp, Artificial Sequence
  Description of Artificial Sequence Synthetic primer
tcttcaagccatcctgtgtg

SEQ ID NO: 64 - DNA, 20 bp, Artificial Sequence
  Description of Artificial Sequence Synthetic primer
atccgcataatctgcatggt

SEQ ID NO: 65 - DNA, 26 bp, Artificial Sequence
  Description of Artificial Sequence Synthetic primer
tctgggacagtagaaagggcttcatc

SEQ ID NO: 66 - DNA, 22 bp, Artificial Sequence
  Description of Artificial Sequence Synthetic primer
actgggcgtggtgtgcttattt

SEQ ID NO: 67 - DNA, 26 bp, Artificial Sequence
  Description of Artificial Sequence Synthetic primer
cctctgtgggtttgcctagtgtttct

SEQ ID NO: 68 - DNA, 29 bp, Artificial Sequence
  Description of Artificial Sequence Synthetic primer
ccctttgctcactgccactctgattattg

SEQ ID NO: 69 - DNA, 19 bp, Artificial Sequence
  Description of Artificial Sequence Synthetic primer
gccctggtgctactcctct

SEQ ID NO: 70 - DNA, 23 bp, Artificial Sequence
  Description of Artificial Sequence Synthetic primer
gcatcgagactatcgctaatgag

SEQ ID NO: 71 - DNA, 24 bp, Artificial Sequence
  Description of Artificial Sequence Synthetic primer
acgaacagcaggctatgggatcaa

SEQ ID NO: 72 - DNA, 27 bp, Artificial Sequence
  Description of Artificial Sequence Synthetic primer
cacgttccaaggtcttcggtagtatgt

SEQ ID NO: 73 - DNA, 24 bp, Artificial Sequence
  Description of Artificial Sequence Synthetic primer
agacctcagtgggaaggtgtttgt

SEQ ID NO: 74 - DNA, 30 bp, Artificial Sequence
  Description of Artificial Sequence Synthetic primer
cttgggtattgtaggagaagaggctgtagg

SEQ ID NO: 75 - DNA, 26 bp, Artificial Sequence
  Description of Artificial Sequence Synthetic primer
ttccctttgcccttctcctccaatac

SEQ ID NO: 76 - DNA, 27 bp, Artificial Sequence
  Description of Artificial Sequence Synthetic primer
ccttgagtctatctgcttgctgccttt

SEQ ID NO: 77 - DNA, 23 bp, Artificial Sequence
  Description of Artificial Sequence Synthetic primer
tttaggcatcgcaaccgcaaagg

SEQ ID NO: 78 - DNA, 28 bp, Artificial Sequence
  Description of Artificial Sequence Synthetic primer
gccagtccatcctgtagtcatcatagtt

SEQ ID NO: 79 - DNA, 26 bp, Artificial Sequence
  Description of Artificial Sequence Synthetic primer
tgctgttaggaagccactatgtctgg

SEQ ID NO: 80 - DNA, 22 bp, Artificial Sequence
  Description of Artificial Sequence Synthetic primer
acacggccaactggaaactgaa

SEQ ID NO: 81 - DNA, 19 bp, Artificial Sequence
  Description of Artificial Sequence Synthetic primer
gatgggcggcggaaaatag

SEQ ID NO: 82 - DNA, 21 bp, Artificial Sequence
  Description of Artificial Sequence Synthetic primer
gcgtggattctgcataatggt

SEQ ID NO: 83 - DNA, 19 bp, Artificial Sequence
  Description of Artificial Sequence Synthetic primer
acagtcgccgcatcttctt

SEQ ID NO: 84 - DNA, 20 bp, Artificial Sequence
  Description of Artificial Sequence Synthetic primer
acgaccaaatccgttgactc

(19) United States
(12) Patent Application Publication
(10) Pub. No.: US 2015/0051212 A1
(43) Pub. Date: Feb. 19, 2015

(54) Compositions And Methods For Inhibiting Drusen

(71) Applicant: Regenerative Research Foundation, Rensselaer, NY (US)

(72) Inventors: Sally Temple Stern, Slingerlands, NY (US); Jeffrey Stern, Slingerlands, NY (US)

(21) Appl. No.: 14/388,131

(22) PCT Filed: Mar. 13, 2013
(86) PCT No.: PCT/US2013/030755

§ 371 Date: Sep. 25, 2014

Related U.S. Application Data
(60) Provisional application No. 61/616,687, filed on Mar. 28, 2012.
(60) Provisional application No. 61/620,210, filed on Apr. 4, 2012.

(51) Int. Cl. A61K 31/506; A61K 31/496; A61K 31/5025

(52) U.S. Cl. CPC A61K 31/506; A61K 31/5025; A61K 31/496
USPC 514/248; 514/252.18; 514/253.06

(57) ABSTRACT

Described herein are methods for inhibiting drusen.

### CROSS-REFERENCE TO RELATED APPLICATIONS

This application claims priority to provisional applications Ser. No. 61/616,687 filed in the United States Patent and Trademark Office on Mar. 28, 2012 and 61/620,210 filed in the United States Patent and Trademark Office on Apr. 4, 2012. The contents of the prior applications are incorporated herein by reference, in their entirety.

### SEQUENCE LISTING

This application includes a Sequence Listing, which is submitted concurrently with the application, via EFS-Web, as an ASCII format text file 174,166 bytes in size, which was created on Mar. 30, 2012 and named 2756215P.txt. The entire contents of the accompanying Sequence Listing is incorporated herein by reference and in its entirety.

### FIELD OF THE INVENTION

The present invention relates to compositions that modulate the formation of drusen, including compositions and agents that inhibit or reduce drusen formation. The invention also related to compositions and methods which use these compositions to modulate (e.g., reduce or inhibit) drusen formation and are useful, e.g., for treating or preventing diseases and disorders associated with abnormal drusen formation, including age-related macular degeneration (AMD). The invention also relates to novel assays, including in vitro, cell-based assays, that can be used to identify drusen-modulating compositions of the invention.

### BACKGROUND

Age-related macular degeneration (AMD) is a highly prevalent disease and the leading cause of irreversible vision loss among patients over 65 in first world countries. J. Ambati et al., Surv. Opthalmol. (2003) 48(3):257-293; N. Congdon et al., Arch. Ophthalmol. (2004) 122(4):477-485. Between 6 million and 10 million Americans are estimated to have AMD, and thousands of new cases are diagnosed each year in the United States alone. For example, see Klein et al., Arch. Ophthalmol. (2011) 129(1):75-80; Friedman et al., Arch. Ophthalmol. (2004) 122(4):564-572.

AMD is a progressive disease. Early stages are characterized by the formation of deposits between the retinal pigment epithelium (RPE) and the underlying choroid, which contains the blood supply to the eye. Typically this early stage of the disorder, known as age-related maculopathy, does not affect day-to-day vision. However, people with drusen deposits may go on to form more advanced forms of AMD. Two forms are generally, referred to as “dry” and “wet” AMD. Approximately 90% of AMD patients suffer from the “dry” form of AMD. Dry AMD is characterized by drusen and atrophy of the RPE layer, which causes vision loss through the loss of macular photoreceptor function. Neovascular or exudative AMD, the “wet” form of the disorder, is characterized by choroidal neovascularization; i.e., abnormal blood vessel growth arising from the capillary layer beneath the RPE. Bleeding and scarring from these blood vessels cause irreversible damage to the photoreceptors, resulting in rapid vision loss. Although more severe, the wet form of AMD is less prevalent; only about 10% of AMD patients suffer from wet AMD. Wet AMD may be treated, moreover, with anti-angiogenic or anti-VEGF agents. No medical treatment is available for the more prevalent dry form of AMD, however, although vitamin supplements with high doses of antioxidant may slow its progression. For review, see Jager et al., N. Engl. J. Med. (2008) 358:2606-2617.

Drusen deposits are widely regarded as the “hallmark lesion” of AMD. Anderson et al., Am. J. Opthalmology (2002) 134:411-431. The appearance of numerous or large confluent drusen strongly correlates with the disease's development and progression. Id. Yet, while they undisputedly play an important role, the exact functional relationship between drusen and AMD remains poorly understood. Electron micrographs have shown RPE exuding cytoplasm from their basal membranes on top of and into drusen, suggesting that drusen may be, at least in part, products of RPE cells. Ishibashi et al., Invest. Ophthalmol. Vis. Sci. (1986) 27(2):184-193; Ishibashi et al., Am. J. Ophthalmol. (1986) 101(3):342-353. Proteomic analysis of harvested human drusen from both normal and AMD-inflicted eyes have identified a number of proteins that differ in levels of expression. Crabb et al., Proc. Natl. Acad. Sci. U.S.A. (2002) 99:14682-14687. Interestingly, a number of oxidative protein modifications were also observed in drusen from AMD patients, suggesting that oxidative injury may contribute to the pathogenesis of AMD and that oxidative protein modification plays a critical role in drusen formation. Id. Indeed, epidemiological studies of diet, environmental and behavioral risk factors have also suggested that oxidative stress may be a contributing factor of AMD. Cai et al., Prog. Retin. Eye Res. (2000) 19:205-221.

Interestingly, many constituents of drusen are also found in pathophysiological deposits associated with other disorders, such as Alzheimer's disease and atherosclerosis; including apoE, complement and amyloid β (“Aβ”) peptides, to name a few. Ding et al. Proc. Natl. Acad. Sci. U.S.A. (2011) 108:E279-E287 (published online Jun. 20, 2011). A number of reports have suggested that Aβ peptide deposition, in particular, may play at least some role in the pathogenesis of AMD. For example, see Ding et al. (2011), supra; Anderson et al., Exp. Eye Res. (2004) 78:243-256; Dentchev et al., Mol. Vis. (2003) 9:184-190; Johnson et al., Proc. Natl. Acad. Sci. U.S.A. (2002) 99:11830-11835; Luibl et al., J. Clin. Invest. (2006) 116:378-385; Isas et al., Invest Ophthalmol. Vis. Sci. (2010) 51:1304-1310; Ding et al., Vision Res. (2008) 48:339-345; Malek et al., Proc. Natl. Acad. Sci. U.S.A. (2005) 102:11900-11905; Burban et al., Agin Cell (2009) 8:162-177. Administration of anti-Aβ antibodies in a mouse model of AMD is reported to partially attenuate the decline in visual function associated with that model. Ding et al. (2008), supra. See also, Ding et al. (2011), supra; U.S. 2009/0022728 by Lin.

Subsequent studies have used the human RPE cell line ARPE-19 to model oxidative stress in vitro and have reported up-regulation of a number of proteins in response thereto. See Weigel et al., Free Radic. Biol. Med. (2002) 33(10):1419-1432; Weigel et al. Free Radic. Biol. Med. (2003) 35(5):465-474; Strunnikova et al., Invest. Ophthalmol. Vis. Sci. (2004) 45(10):3767-3777. See also, Glotin et al., Free Radic. Biol. Med. (2008) 44:1348-1361. However, studies using these ARPE-19 cells have not been able to demonstrate increased expression of many of the most highly up regulated drusen related proteins identified by Crabb et al., supra. Hence, there remains an ongoing need for in vitro, cell-based models and other assays to study drusen formation and its role in the progression and development of drusen-associated disorders, such as AMD. There also remains an ongoing need for assays that can be used to identify compounds and other agents that modulate (e.g., reduce or inhibit) drusen formation and may be useful for treating drusen-associated disorder such as AMD.

### SUMMARY OF THE INVENTION

The present invention is based, in part, on the discovery that a recently discovered retinal pigment epithelial stem cell (RPESC) can be differentiated into mature retinal pigment epithelial (RPE) cells. See Salero et al. Cell Stem Cell (Jan. 6, 2012) 10(1):1-2; see also U.S. Patent Application Publication No. 2009/0274667 Al and International Patent Application Publication No. WO 2009/132156. The invention is also based in part on the discovery RPE cells derived from such RPESC may be used in vitro to study drusen formation and, in particular, to identify compounds and other compositions that modulate drusen formation in such cells. More specifically, the invention is based in part on the discovery that the expression of proteins associated with drusen formation, and also of genes encoding such proteins, is upregulated in the RPESC-derived RPE cells after subjecting the RPE cells to conditions of oxidative stress, e.g., by exposing them to oxidative agents such as hydrogen peroxide (H₂O₂) or tert-butyl hydroperoxide (TBHP). Such drusen-associated genes and proteins include, but are not limited to, the exemplary drusen-associated genes listed in Table I, infra, as well as their gene products—e.g., polypeptides encoded by such drusen-associated genes.

The invention is also based, in part, on the identification of compounds that inhibit the upregulation of such drusen-associate genes in proteins in the assays of this invention. Such compounds include, inter alia, the compound imatinib and pharmaceutically acceptable salts thereof, such as the mesylate salt. Such compounds are therefore useful, in the novel methods and compositions of these invention, for modulating drusen formation in vitro or in vivo, including methods and compositions for inhibiting or reducing the formation of drusen in vitro or in vivo. Compounds identified in the methods of this invention, including imatinib and pharmaceutically acceptable salts thereof, can also be used in novel therapeutic methods, e.g., to reduce or inhibt drusen formation in a subject, and therefore ameliorate, prevent or inhibit drusen-associated disorders such as age-related macular degeneration (AMD).

In conjunction with these discoveries, described herein are methods for inhibiting drusen in a subject comprising administering to a subject in need thereof (e.g., a patient (e.g., a mammal, preferably, a human) suffering from or at risk of developing dry age related macular degeneration (AMD) a composition comprising an inhibitor of one or more, preferably two or more, and most preferably all of the polypeptides selected from the group consisting of gamma secretase activating protein (GSAP), platelet derived growth factor receptor (PDGFR) and c-Abl tyrosine kinase (BRC-Abl) and up-regulators of neprilysin. Preferably, the composition comprises an effective amount for inhibiting drusen of imatinib mesylate.

Preferably, inhibition of drusen comprises inhibiting the expression of one or more drusen-associated polypeptides, e.g., amyloid protein beta (A(β), amyloid precursor protein (APP), apolipoprotein E (APOE), apolipoprotein J (APOJ), αβ-crystallin, β-site AβPP cleaving enzyme 1 (BACE-1), presenilin 1 (PS1), and vascular endothelial growth factor (VEGF)-A. In certain instances, the method can comprise administering imatinib mesylate in combination with another drug or therapy (i.e., a combination therapy) for inhibiting drusen. For example, imatinib mesylate may be co-administered with a small molecule inhibitor of drusen and/or of amyloid beta (Aβ) and/or an antibody that is specific for a drusen-associated protein (e.g., Aβ, APP, BACE-1, etc.). In some instances, imatinib mesylate (or related compound) is directly conjugated to the drusen-associated protein-specific antibody.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488- 492 (1985), U.S. Pat. No. 5,071, 743, Fukuoka et al., Biochem. Biophys. Res. Commun. 263: 357-360 (1999); Kim and Maas, BioTech. 28: 196-198 (2000); Parikh and Guengerich, BioTech. 24: 428-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nuc. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218. The skilled person will know and be able to use these and other techniques routine in the art to practice the present invention.

Other features and advantages of the invention will be apparent from the following detailed description and figures, and from the claims.

### BRIEF DESCRIPTION OF THE DRAWINGS

FIG. 1 is a bar graph quantifying the fold induction of the indicated drusen-associated proteins over control in an RPE cell based in vitro model of drusen formation. The “*” indicates statistical significance as determined by the Student's t test (p<0.05).

FIG. 2 is a bar graph quantifying the percent (%) cell death as determined by LDH assay in adult human RPE cells subjected to oxidative stress and treated with the indicated doses of imatinib mesylate (“Gleevec”), DAPT, or ponatinib.

FIG. 3 is a bar graph quantifying the transepithelial resistance (Ω·cm²) of stressed RPE cells following treatment with the indicated doses of imatinib mesylate (“Gleevec”), DAPT, ponatinib or bosutinib, or vehicle control.

FIG. 4 is a bar graph quantifying the mRNA expression level (fold induction over control) of the indicated drusen-associated proteins.

### DETAILED DESCRIPTION

The present invention is based, at least in part, on the discovery that retinal pigment epithelial (RPE) cells derived from an RPE stem cell (RPESC) may be used in vitro to study drusen formation and, in particular, to identify compounds and other compositions that modulate drusen formation in such cells. More specifically, the invention is based in part on the discovery that the expression of proteins associated with drusen formation, and also of genes encoding such proteins, is upregulated in the RPESC-derived stems cells after subjecting the cells to conditions of oxidative stress, e.g., by exposing them to oxidative agents such as hydrogen peroxide (H₂O₂) or tert-butyl hydroperoxide (TBHP). Such drusen-associated genes and proteins include, but are not limited to, the exemplary drusen-associated genes listed in Table I, infra, as well as their gene products—e.g., polypeptides encoded by such drusen-associated genes.

The invention is also based, in part, on the identification of compounds that inhibit the upregulation of such drusen-associate genes in proteins in the assays of this invention. Such compounds include, inter alia, the compound imatinib and pharmaceutically acceptable salts thereof, such as the mesylate salt. Such compounds are therefore useful, in the novel methods and compositions of these invention, for modulating drusen formation in vitro or in vivo, including methods and compositions for inhibiting or reducing the formation of drusen in vitro or in vivo. Compounds identified in the methods of this invention, including imatinib and pharmaceutically acceptable salts thereof, can also be used in novel therapeutic methods, e.g., to reduce or inhibt drusen formation in a subject, and therefore ameliorate, prevent or inhibit drusen-associated disorders such as age-related macular degeneration (AMD).

### Drusen

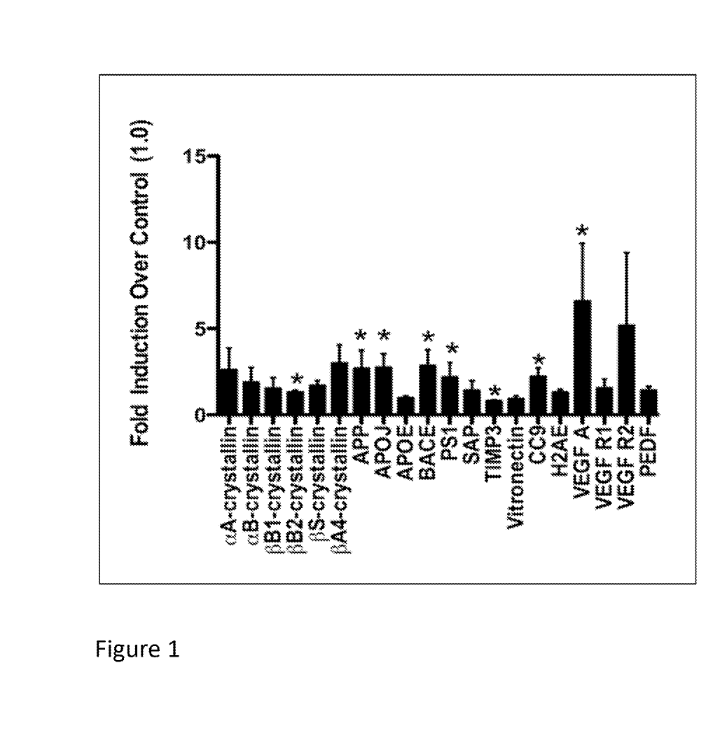

Drusen are small, extracellular deposits that form between the retinal pigment epithelium (RPE) and the underlying Bruch's membrane, which separates the RPE from the choriocapillaria that supply blood to the eye. They are visible in an ophthalmoscopic examination as focal yellow dots. See, for example, FIG. 1 of Williams, Age and Ageing (2009) 38:468-654.

Drusen deposits contain a number of different denatured proteins and protein fragments, as well as lipids, carbohydrates and trace elements such as zinc. Protein components include, without limitation, apolipoproteins and members of the complement system, such as complement factor H. Other, non-limiting examples of drusen-associated proteins include αA-crystallin, αB-crystallin, βB1-crystallin, βB2-crystallin, βs-crystallin, αA4-crystallin, Aβ. APP, APOE, APOJ, BACE-1, PS1, VEGF, VEGF R1, VEGF R2, PEDF, CC9, serum amyloid P, TIMP3, vitronectin, as well as fragments and derivatives of those proteins that may be created, e.g., by oxidative damage. Exemplary sequences for these drusen-associated proteins, and/or for genes encoding them, are known in the art and include, by way of example, the sequences identified in Table 1 below, by their GenBank® Accession numbers.

TABLE 1
EXEMPLARY DRUSEN ASSOCIATED SEQUENCES

| | Nucleotide Sequence | | Amino Acid Sequence | |
|---|---|---|---|---|
| | | SEQ | | SEQ |
| | GenBank | ID | GenBank | ID |
| Name | Accession No. | NO. | Accession No. | NO. |
| complement | NM_000186.3 | 1 | NP_000177.2 | 2 |
| factor H | | | | |
| αA-crystallin | NM_000394.2 | 3 | NP_000385.1 | 4 |
| αB-crystallin | NM_001885 | 5 | NP_001876.1 | 6 |
| βB1-crystallin | NM_001887.3 | 7 | NP_001878.1 | 8 |
| βB2-crystallin | NM_000496 | 9 | NP_000487.1 | 10 |
| βs-crystallin | NM_017541.2 | 11 | NP_060011.1 | 12 |
| αA4-crystallin | NM_001886.2 | 13 | NP_001877.1 | 14 |
| amyloid precursor | NM_000484.3 | 15 | NP_000475.1 | 16 |
| protein (APP) | | | | |
| APOE | NM_001128917.1 | 17 | NP_001122388.1 | 18 |
| APOJ | NM_001831.3 | 19 | NP_001822.3 | 20 |
| BACE-1 | NM_138973.3 | 21 | NP_620429.1 | 22 |
| PS1 | NM_000021.3 | 23 | NP_000012.1 | 24 |
| VEGF | NM_001025366 | 25 | NP_001020537.2 | 26 |
| VEGF R1 | NM_002019.4 | 27 | NP_002010.2 | 28 |
| VEGF R2 | NM_002253.2 | 29 | NP_002244.1 | 30 |
| PEDF | NM_002615 | 31 | NP_002606.3 | 32 |
| CC9 | NM_001737 | 33 | NP_001728.1 | 34 |
| serum amyloid P | NM_001639.3 | 35 | NP_001630.1 | 36 |
| TIMP3 | NM_000362.4 | 37 | NP_000353.1 | 38 |
| Vitronectin | NM_000638.3 | 39 | NP_000629.3 | 40 |

### RPE Cell-Based Screening Assay

In the present invention, compounds and other compositions that modulate or affect drusen formation are preferably identified using in vitro cell-based models and assays for drusen formation, such as those described in the Examples, infra. Such models and assays preferably use cells or cell lines that are derived from a retinal pigment epithelial stem cell (RPESC). In preferred embodiments, the models and assays of this invention make use of retinal pigment epithelial (RPE) cells that are derived from the RPESC.

Cells used in the assays and models of the invention overexpress drusen-associated polypeptides and/or genes encoding the same when subject to conditions of oxidative stress, such as treating the cells with hydrogen peroxide (H₂O₂) or with tert-butyl hydroperoxide (TBHP). Preferred drusen-associated polypeptides and genes include those identified, supra, including, without limitation, drusen-associated polypeptides and genes comprising the sequences identified in Table 1, supra, by their GenBank® accession numbers. Specific, preferred examples include, without limitation, aβ2-crystallin, Aβ, APP, APOJ, APOE, BACE-1, PS1, TMP3, CC9 and VEGF.

Generally speaking, assays of the invention involving culturing RPE derived from the RPESC under conditions of oxidative stress (e.g., in the presence of H₂O₂, TBHP or another oxidative reagent). The cells may then be incubated with a test compound, either before, after or during their exposure to the oxidative stress conditions, and the expression of one or more drusen-associate polypeptides and/or genes is determined Those expression levels may then be compared to one or more controls, and are preferably compared to expression levels of the same drusen-associated polypeptides and/or genes in the same cell line that are cultured under identical conditions of oxidative stress but without exposure to the test compounds. Preferably, the expression levels are also compared to expression levels of the same drusen-associated polypeptides and/or genes in cells of the same cell line that are not cultured under conditions of oxidative stress. Where, in cells incubated with the test compound, the determined expression levels, under the oxidative stress conditions, of the one or more drusen-associated polypeptides and/or genes are down regulated, compared to their expression levels, under the oxidative stress conditions, in cells that are not incubated with the test compound, the compound is thus identified as a compound which affects drusen formation and, in particular, as a compound that reduces or inhibits drusen formation. In preferred embodiments, the test compound is identified as a compound that reduces or inhibits drusen formation when determined expression levels of the one or more drusen-associated polypeptides and/or genes under oxidative stress conditions, in the cells incubated with that test compound, are identical or substantially similar to their expression levels in cells not cultured under conditions of oxidative stress.

### RPE Stem Cells (RPESCs)

Methods for isolation and culture of RPESCs are known and described in detail, e.g., in U.S. Patent Application Publication No. 2009/0274667 by Temple et al. See also, Salero et al., Cell Stem Cell (2012) 10:88-95. In the in vitro RPE cell-based assay, isolated RPE cells are activated to a stem cell state, the RPESC, and then cultured to produce diverse progeny. Some of the RPESC progeny are pathologic in that they over-express drusen proteins. De et al., Arch Ophthalmol. (2007) 125:641-646; and Salero et al. Cell Stem Cell (2012) 10:88-95. Such pathologic, drusen protein-expressing RPESC progeny serve as a “disease-in-a-dish” model for drusen formation in RPE cells, and the RPE screening assay described in the present Examples can thus be used to characterize one or more effects of a test compound on the RPE cells, and, in particular, to identify candidate drusen inhibitors.

The pathologic, drusen-forming progeny RPE cells of the assay can be treated in conditions, such as, e.g., induction of cellular stress using, e.g., tert-butyl hydroperoxide (TBHP) or H₂O₂, that cause further upregulation of drusen-associated polypeptides, as well as affect other characteristics of the cells that are associated with drusen formation and/or AMD. Such characteristics may include, for example, cell viability and epithelial integrity, and then the ability of a test substance to inhibit one or more of the characteristics induced by the cellular stress (e.g., upregulated drusen-associated polypeptide expression levels, cell death, and decreased epithelial integrity) can be determined However, in certain instances, the RPE cells can be used in the screening assay to identify candidate drusen inhibitors without first inducing artificial cellular stress because there is a low level of background oxidative stress normally present in the culture media that is sufficient to induce a low level of expression of drusen protein and other characteristics of drusen and AMD .

Preferably, although not necessarily, a candidate compound identified by the present RPE assay inhibits 1 or more, or 2 or more, of the characteristics of the drusen-protein expressing RPE cells (e.g., decreased viability, decreased epithelial integrity, and increased drusen protein expression). For example, a candidate compound may inhibit expression of one or more drusen-associated polypeptides (i.e., is an inhibitor of drusen) and/or one or more other characteristics of RPE cells of the assay (e.g., decreased viability and decreased epithelial integrity). In some embodiments, the RPE cells may be treated with an inducer of cellular stress (e.g., H202, TBHP or Aβ). Most preferably, although not necessarily, an inhibitor identified as an inhibitor of drusen may also inhibit cell death and loss of epithelial integrity, e.g., as induced, e.g., by cellular stress and/or by the presence of drusen or drusen-like deposits.

Expression levels of a drusen-associated polypeptide in the cultured RPE cells can be determined according to any suitable method known in the art, such as, e.g., quantitative, real-time reverse transcriptase polymerase chain reaction (qPCR) or Northern blot (to quantify mRNA expression), and/or immunocytochemical staining, Western blot, and/or ELISA (to quantify protein expression). For example, the expression or level of such proteins can be detected using immunohistochemistry, immunofluorescence, Western blotting, protein chip technology, immunoprecipitation, ELISA assay, or mass spectrometry using standard methods known in the art. These methods can be performed using antibodies or antigen-binding antibody fragments that specifically bind to that mammalian (e.g., human) protein. Detection using these antibodies or antigen-binding antibody fragments can be facilitated by coupling the antibody or antigen-binding antibody fragment to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Preferably, a test substance may be identified in the screening assay as a drusen inhibitor, if the expression level one or more of the drusen-associated polypeptides described herein (e.g., aβ2-crystallin, Aβ, APP, APOE, APOJ, BACE-1, PS1, TMP3, CC9 and VEGF A) is decreased following treatment of the cells with the test substance. More preferably, the expression level of 2 or more, 3 or more, 4 or more, 5 or more, 6 or more, 7 or more, or 8 or more drusen-associated polypeptides is decreased, compared to the level in the absence of treatment with the test substance.

In another embodiment, a test substance may be identified as an inhibitor of drusen if the test substance decreases the upregulated expression level of one or more of the drusen-associated polypeptides described herein (e.g., as aβ2-crystallin, Aβ, APP, APOE, APOJ, BACE-1, PS1, TMP3, CC9 and VEGF A) resulting from induction of cellular stress (e.g., with TBHP, H2O2 or Aβ). More preferably, the upregulated expression levels of 2 or more, 3 or more, 4 or more, 5 or more, 6 or more, 7 or more, or 8 or more drusen-associated polypeptides are decreased, compared to the levels in the absence of treatment with the test substance.

Preferably, for a test substance that is identified as an inhibitor of drusen, the upregulated expression level of one or more drusen-associated polypeptides (e.g., mRNA and/or protein level) that results from induction of cellular stress is decreased relative to the negative control (e.g., no treatment or treatment with a vehicle) by at least 1.5-fold, at least 2-fold, at least 3-fold, at least 4-fold, at least 5-fold, at least 6-fold, at least 10-fold, or more.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids. In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art and/or described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein. In some embodiments, a monoclonal antibody directed against a polypeptide can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP* Phage Display Kit, Catalog No. 240612). Additionally, examples of methods and reagents particularly amenable for use in generating and screening antibody display library can be found in, for example, U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology 9:1370-1372, 1991; Hay et al., Hum. Antibod. Hybridomas 3:81-85, 1992; Huse et al., Science 246:1275-1281, 1989; Griffiths et al., EMBO J. 12:725-734, 1993. In some embodiments, the antigen-binding antibody fragment is a Fab fragment, a F(ab′)2 fragment, and a scFv fragment. Methods f or generating these antibody fragments are known in the art.

Included herein are methods for screening test compounds, e.g., polypeptides, polynucleotides, inorganic or organic large or small molecule test compounds, antibodies, to identify agents which are inhibitors of drusen formation. As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da). The test compounds can be natural products, known therapeutics, small molecules, nucleic acids, or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Cell viability can be measured according to any suitable method. For example, cell death can be measured using an LDH assay, which measures the amount of lactate dehydrogenase released from dead and dying cells. Other assays for cell viability include measuring Annexin V expression. Typically, a test substance may be identified as a candidate inhibitor of drusen in the assay, if the test substance restores cell viability in RPE cells overexpressing drusen proteins (e.g., following induction of cellular stress). Restoration of cell viability can be determined, e.g., by measuring decreases in cell death compared to a control (e.g., cell lysate or RPE cells that had been treated with a cell stress inducer (e.g., TBHP), but no test substance). Preferably, cell death is decreased, relative to the control, by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% (i.e., completely).

The ability of a test substance to restore transepithelial resistance may also be determined in the RPE cell-based assay. RPE cells are a tight junctioned epithelia and compromised epithelial integrity is a hallmark of RPE-associated diseases, such as, e.g., dry AMD. Healthy epithelial cells exhibit a high resistance measurement that can be easily experimentally assessed. Transepithelial resistance can be measured using, e.g., the WPI EVOM Voltohmmeter protocol (described by the manufacturer of the volt-ohm meter available from World Precision Instruments (Sarasota, FL). Typically, in the assay, the RPE cells are permitted to reach a resistance of >200 Ω·cm² prior to beginning the experiment. Compromised cells (i.e., poor epithelial integrity) will typically have a resistance of less than 200 Ω·cm². Table 2 below shows exemplary compounds identified by screening assays of the invention, which may be useful in methods of the invention, e.g., for treating AMD and/or inhibiting drusen formation.

TABLE 2
EXEMPLARY CANDIDATE COMPOUNDS

| | Restored | | Reduced | Clinical | Mechanism | |
|---|---|---|---|---|---|---|
| | Resis- | Restored | Drusen | Trial | of | |
| Drug | tance | Viability | Proteins | Phase | Action | Structure |
| Gleevec 0.1 μM Gleevec 1 μM Gleevec 10 μM | − + − | + + + | − + unknown | I, II, III, IV | Inhibits γ-seecretase activating protein, c- Abl, and PDGF-R | 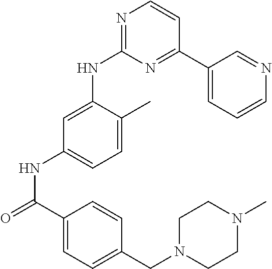 [SMILES: Cc1ccc(NC(=O)c2ccc(CN3CCN(C)CC3)cc2)cc1Nc1nccc(-c2cccnc2)n1] |
| DAPT 0.1 μM DAPT 1 μM DAPT 10 μM | − + − | − − − | + + | None | Inhibits secretase | 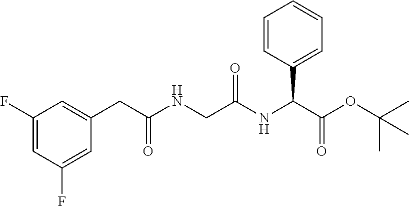 [SMILES: CC(C)(C)OC(=O)[C@@H](NC(=O)CNC(=O)Cc1cc(F)cc(F)c1)c1ccccc1] |
| Ponatinib 0.1 μM Ponatinib 0.5 μM Ponatinib 1.0 μM | − − − | + − − | + + − | I, II | Inhibits c-Abl | 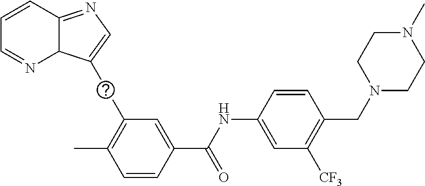 [SMILES: Cc1ccc(C(=O)Nc2ccc(CN3CCN(C)CC3)c(C(F)(F)F)c2)cc1CC1=CN=C2C=CC=NC12] |
| Bosutinib 0.1 μM Bosutinib 1 μM | − − | unknown unknown | + + | I, II, III | Inhibits c-Abl | 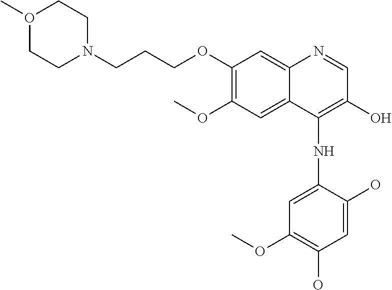 |
| Phenserine μM | − | − | unknown | I, II | Post- tran- scriptional inhibition of APP synthesis | 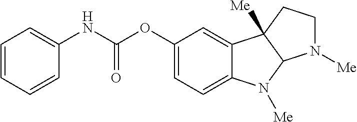 [SMILES: CN1CC[C@@]2(C)c3cc(OC(=O)Nc4ccccc4)ccc3N(C)C12] |
| Posiphen 1 μM | − | + | unknown | I | Post- tran- scriptional inhibition of APP synthesis | 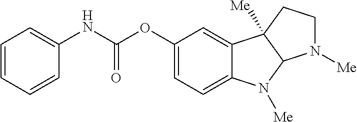 [SMILES: CN1CC[C@]2(C)c3cc(OC(=O)Nc4ccccc4)ccc3N(C)C12] |
| N1- norphenserine 1 μM | + | + | unknown | None | Post- tran- scriptional inhibition of APP synthesis | 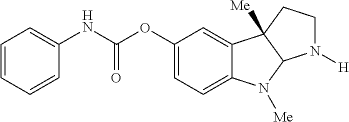 [SMILES: CN1c2ccc(OC(=O)Nc3ccccc3)cc2[C@]2(C)CCNC12] |
| Bisnorphenserine 1 μM | + | + | unknown | None | Post- tran- scriptional inhibition of APP synthesis | 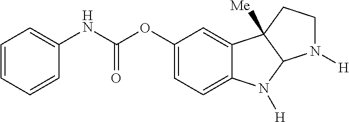 [SMILES: C[C@@]12CCNC1Nc1ccc(OC(=O)Nc3ccccc3)cc12] |
| N8- norphenserine 1 μM | + | + | unknown | None | Post- tran- scriptional inhibition of APP synthesis | 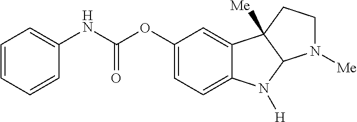 [SMILES: CN1CC[C@@]2(C)c3cc(OC(=O)Nc4ccccc4)ccc3NC12] |

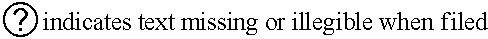

In some embodiments, a test compound is applied to a test sample, e.g., a cell or living tissue or organ (e.g., an eye) and one or more effects of the test compound is evaluated. In a cultured or primary cell for example, the ability of the test compound to decrease the expression levels of one or more of the drusen associated polypeptides following treatment of the cells with the test sample. Further, in a cultured or primary cell for example, the ability of the test compound to decrease the expression levels of one or more of the drusen associated polypeptides resulting from induction of oxidative stress following treatment of the cells with the test sample.

In some embodiments, the test sample is, or is derived from (e.g., a sample taken from) an in vivo model of a disorder as described herein. For example, an animal model, e.g., a rodent such as a rat, can be used.

A test compound that has been screened by a method described herein and determined to affect drusen associated proteins, can be considered a candidate compound. A candidate compound that has been screened, e.g., in an in vivo model of a disorder, e.g., AMD and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” e.g., test compounds that show a marked decreased in drusen associated proteins in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating disorders associated with the eye as described herein, e.g., AMD. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to cell model of a disorder described herein e.g. AMD. The cell line (or tissue) can be monitored for a change in the disorder, e.g., for an improvement in a parameter of the disorder, e.g., a parameter related to clinical outcome. In some embodiments, the parameter is viability of the cell, and an improvement would be decreased cell death. In some embodiments, the parameter is epithelial integrity or resistance, and an improvement would be increased integrity or resistance of the epithelial cell layer.

Test compounds identified as candidate therapeutic compounds can be further screened by administration to an animal model of a disorder described herein. The animal can be monitored for a change in the disorder, e.g., for an improvement in a parameter of the disorder, e.g., a parameter related to clinical outcome. In some embodiments, the parameter may be reduced appearance of drusen, and an improvement may be be decreased leakage from blood vessels or decreased atrophy of the RPE layer. In some embodiments, the subject is a human, e.g., a human with AMD. In such embodiments, the parameter used may be, e.g., reduced drusen and an improvement may be decreased leakage from blood vessels or decreased atrophy of the RPE layer.

### Imatinib Mesylate

As demonstrated in Example 2, imatinib mesylate (also known as Gleevec® (Novartis Pharmaceuticals, East Hanover, N.J.)) was identified using the RPE cell-based screening assay described, above, as an inhibitor of drusen. Imatinib mesylate is approved by the FDA for use in humans for the treatment of cancer.

Imatinib mesylate has the formula C₂₉H₃₁N₇O and the IUPAC name 4-[(4-methylpiperazin-1-yl)methyl]-N-(4-methyl-3-{[4-(pyridin-3 -yl)pyrimidin-2-yl]amino}phenyl)benzamide. Imatinib mesylate is described in detail in U.S. Pat. No. 6,894,051. The free form, imatinib, is also encompassed by the present invention.

Imatinib mesylate-like compounds and imatinib mesylate-related compounds (e.g., derivatives), as well as different crystalline forms (e.g., beta form described in U.S. Pat. No. 6,894,051) may be used to inhibit drusen, as described herein. Such compounds are known and described in the art. For example, AMN107 is a modified form of imatinib mesylate that is 20-fold more potent that imatinib mesylate. (See, O'Hare et al. 2005 Cancer Cell; 7:117-119.) Other examples include BMS-354825 and ON012380, also described in O'Hare et al. Other derivatives of imatinib mesylate can be designed by one of skill in the art, and such derivatives are also encompassed herein. For example, the piperazine moiety of imatinib mesylate can be modified to improve binding.

The skilled artisan can readily determine whether an imatinib mesylate related compound is encompassed by the present invention, e.g., by testing the compound in the RPE screening assay described above. For example, an imatinib mesylate-related compound encompassed by the present invention will decrease the expression level of at least one drusen-associated polypeptide (e.g., at least one of aβ2-crystallin, Aβ, APP, APOE, APOJ, BACE-1, PS1, TMP3, CC9 and VEGF A) in the RPE assay compared to a control group (e.g., no test compound). Further, an imatinib mesylate-related compound and/or imatinib mesylate derivative will preferably inhibit one or more, more preferably two or more, and most preferably all of the polypeptides selected from the group consisting of gamma secretase activating protein (GSAP), platelet derived growth factor receptor (PDGFR), and c-Abl tyrosine kinase (BRC-Abl) to inhibit APP and Aβ production and upregulation of neprilysin to increase abeta breakdown

Modified, crystalline imatinib mesylate compounds and/or imatinib mesylate-related compounds are described e.g., in U.S. Pat. No. 6,894,051 to Zimmermann.

Other drugs (e.g., small molecules) that may or may not be related to (i.e., have a similar chemical structure) or derived from imatinib mesylate are also encompassed by the present invention, provided that they inhibit one or more, more preferably two or more, and most preferably all of the polypeptides selected from the group consisting of gamma secretase activating protein (GSAP), platelet derived growth factor receptor (PDGFR) and c-Abl tyrosine kinase (BRC-Abl). Further, such drugs preferably decrease the expression level of at least one drusen-associated polypeptide (e.g., at least one of aβ2-crystallin, Aβ, APP, APOE, APOJ, BACE-1, PS1, TMP3, CC9 and VEGF A), e.g., in the RPE assay described herein, compared to a control group (e.g., no drug). Also, the upregulation of drugs that accelerate Aβ βreakdown such as up-regulates neprilysin are considered.

### Pharmaceutical Formulations

While the compositions of the invention may be administered alone, in certain embodiments, it may be preferable to formulate the composition in combination with a pharmaceutically acceptable carrier.

As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “pharmaceutically acceptable derivative” as used herein means any pharmaceutically acceptable salt, solvate or prodrug, e.g., ester, of a compound of the invention, which upon administration to the recipient is capable of providing (directly or indirectly) a compound of the invention, or an active metabolite or residue thereof Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. Preferred pharmaceutically acceptable derivatives are salts, solvates, esters, carbamates, and phosphate esters. Particularly preferred pharmaceutically acceptable derivatives are salts, solvates, and esters. Most preferred pharmaceutically acceptable derivatives are salts and esters.

A pharmaceutical composition is formulated to be compatible with its intended route of administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection or infusion typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition should be sterile and should be fluid to the extent that easy syringability exists. Pharmaceutical formulations are ideally stable under the conditions of manufacture and storage and should be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be advantageous to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent (e.g., imatinib mesylate) in the required amount in an appropriate solvent with one or a combination of ingredients enumerated above, as required, followed by filtered sterilization. Generally, dispersions are prepared by incorporating the purified antibody or antigen binding fragment into a sterile vehicle which contains a basic dispersion medium and the required other ingredients from those enumerated above. In the case of sterile powders for the preparation of sterile injectable solutions, exemplary methods of preparation are vacuum drying and freeze-drying which yields a powder of the active ingredient plus any additional desired ingredient from a previously sterile-filtered solution thereof

Oral compositions generally include an inert diluent or an edible carrier. For the purpose of oral therapeutic administration, the active agent (e.g., imatinib mesylate) can be incorporated with excipients and used in the form of tablets, troches, or capsules, e.g., gelatin capsules. Oral compositions can also be prepared using a fluid carrier for use as a mouthwash.

For administration by inhalation, the pharmaceutical formulation is preferably delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the purified polypeptide or protein and delivery agents are formulated into ointments, salves, gels, or creams as generally known in the art.

In certain embodiments, compositions are prepared with carriers that will protect the active agent (e.g., imatinib mesylate) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, the disclosure of which is incorporated herein by reference in its entirety.

Pharmaceutically compatible binding agents, and/or adjuvant materials can be included as part of the formulation. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

Formulations for intraocular administration can include formulation suitable for injection or infusion described above which typically include sterile aqueous solutions (where water soluble) or dispersions and sterile powders for the extemporaneous preparation of sterile injectable solutions or dispersion. For intravitreous administration, suitable carriers include physiological saline, bacteriostatic water, PLGA, Cremophor ELTM (BASF, Parsippany, N.J.) or phosphate buffered saline (PBS). For review, see, Kuno & Fujii, Polymers (2011) 3:193-221. In all cases, the composition should be sterile and should be fluid to the extent that easy syringability exists. Further, a composition can be adapted to be used as eye drops, or injected into or around the eye, e.g., using peribulbar or intravitreal injection. Such compositions should be sterile and substantially endotoxin-free, and within an acceptable range of pH. Certain preservatives are thought not to be good for the eye, so that in some embodiments a non-preserved formulation is used. Formulation of eye medications is known in the art, see, e.g., Ocular Therapeutics and Drug Delivery: A Multi-Disciplinary Approach, Reddy, Ed. (CRC Press 1995); Kaur and Kanwar, Drug Dev Ind Pharm. 2002 May;28(5):473-93; Clinical Ocular Pharmacology, Bartlett et al. (Butterworth-Heinemann; 4th edition (March 15, 2001)); and Ophthalmic Drug Delivery Systems (Drugs and the Pharmaceutical Sciences: a Series of Textbooks and Monographs), Mitra (Marcel Dekker; 2nd Rev&Ex edition (Mar. 1, 2003)).

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent (e.g., imatinib mesylate) calculated to produce the desired therapeutic effect in association with the required pharmaceutical carrier.

### Methods of Treatment

Described herein are methods for inhibiting drusen (e.g., decreasing drusen deposits and/or inhibiting drusen deposits). The presence of drusen deposits in the RPE is the hallmark feature of dry AMD. Thus dry AMD can be treated according to the method described herein.

While not intending to be bound by one particular theory or mechanism of action, the treatment methods described herein are thought to treat drusen, at least in part, by reducing the expression level of one or more drusen-associated polypeptides (e.g., APP, Aβ, APOE, APOJ, αβ-crystallin, BACE1, PS1, VEGF-A), thereby reducing drusen biosynthesis and/or the size and/or number of existing drusen and/or drusen-like deposits. Thus, efficacy of treatment with a composition or formulation described herein in a subject suffering from or at risk of developing a drusen-associated disease or condition (e.g., dry AMD) can be determined, e.g., by quantifying the number of drusen (and/or drusen-like) deposits at the affected site (e.g., RPE layer of retina) and/or by measuring the size of one or more drusen and/or drusen-like deposits, wherein, preferably, the treatment reduces the size and/or number of drusen and/or drusen-like deposits at the affected site.

Preferably, the size and/or number of drusen and/or drusen-like deposits at the affected site (e.g., RPE) is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% (i.e., completely), compared to the size and/or number of drusen and/or drusen-like deposits prior to or at the beginning of the treatment.

### Administration and Dosage

Compositions and formulations can be administered topically, intraocularly, parenterally, orally, by inhalation, as a suppository, or by other methods known in the art. The term “parenteral” includes injection (for example, intravitreous, intravenous, intraperitoneal, epidural, intrathecal, intramuscular, intraluminal, intratracheal or subcutaneous). A preferred route of administration is intraocular, for example by intravitreous injection.

Compositions may be administered as needed, e.g., once a day, twice a day, or more often. However, those skilled in the art will appreciate that it is preferable to minimize the frequency of administration needed to obtain a therapeutic effect, to minimize or reduce the risk of damage, e.g., from excessive intravitreous injection into a patient's eye. Frequency may be decreased during a treatment maintenance phase of the disease or disorder, e.g., once every second or third day instead of every day or twice a day. The dose and the administration frequency will depend on the clinical signs (e.g., presence or absence or decreased levels of drusen or drusen-like deposits).

It will be appreciated that the amount of active agent (e.g., imatinib mesylate or related compound) required for use in treatment will vary with the route of administration, the nature of the condition for which treatment is required, and the age, body weight and condition of the patient, and will be ultimately at the discretion of the attendant physician or veterinarian. Compositions will typically contain an effective amount (e.g., therapeutically effective amount) of the active agent(s), alone or in combination. Preliminary doses can be determined according to animal tests, and the scaling of dosages for human administration can be performed according to art-accepted practices.

The initial dose may be larger, followed by smaller maintenance doses. The dose may be administered, e.g., weekly, biweekly, daily, semi-weekly, etc., to maintain an effective dosage level. An effective amount of the composition may be the amount administered after a single administration, or may be the total amount administered over a plurality of administrations, e.g., two or more, three or more, four or more, five or more, six or more, seven or more, eight or more, nine or more, ten or more, etc., administrations of the composition.

Therapeutically effective dosages can be determined stepwise by combinations of approaches such as (i) characterization of effective doses of the composition or compound in in vitro cell culture assays, e.g., using the RPE cell-based model described herein (see, e.g., Example 1) in which drusen protein expression and/or cell viability is used as a readout, followed by (ii) characterization in animal studies using drusen formation as a readout, followed by (iii) characterization in human trials using decreased drusen formation (e.g., in the RPE) as a readout.

Exemplary dosages of imatinib mesylate, e.g., for administration in tablet form, are 400-800 mg/day for adult patients. A starting dose of 100 mg/day may be considered for some patients, and the dose may then be subsequently increased, depending on the response and the experience of side effects. Intraocular dosages of 1, 10, 100 and 1000 micrograms are contemplated with the preferred dose of 10 or 100 microgrqams.

### Combination Therapy

The drusen-inhibitors of the invention (e.g., imatinib mesylate and its related compounds) can be administered in combination with other therapeutic agents, e.g., for the treatment of drusen. For example, a drusen inhibitor can be administered with an agent that reduces amyloid beta (Aβ), a peptide which has been found to be associated with drusen formation, or other drusen-associated polypeptides.

In certain embodiments, a drusen inhibitor can be co-administered with a therapeutic agent that increases cellular integrity and/or reduces the effect of reactive oxygen species and/or other cell damage associated with amyloid and/or amyloid-like deposits such as drusen. In certain embodiments, a drusen inhibitor described herein can be co-administered with a Bcr-Abl inhibitor, such as, e.g., ponatinib, bosutinib, dasatinib or PD180970 (La Rosee et al. 2002; Cancer Res 62:7149-7153), or retinoid X receptor agonists such as bexarotene that increase APP and Aβ clearance, or an inhibitor of γ-secretase APP/Notch (e.g., DAPT), or sunitinib (a PDGFR inhibitor). Such inhibitors are available commercially, e.g., from Axon Medchem (Netherlands).

In another embodiment, a drusen inhibitor (e.g., imatinib mesylate or related compound) can be directly conjugated to another agent. The other agent can be another therapeutic agent (e.g., small molecule drug or antibody). In a preferred embodiment, the drusen inhibitor is conjugated to an antibody that specifically binds to amyloid beta (Aβ) or another polypeptide found in drusen deposits (e.g., αβ2-crystallin, Aβ, APP, APOE, APOJ, BACE-1, PS1, TMP3, CC9 or VEGF A). The antibody may be a therapeutic antibody (e.g., one that itself inhibits drusen, e.g., by promoting clearance of deposits and/or by decreasing expression of one or more drusen-associated polypeptides) and/or may aid in targeting the drusen inhibitor (e.g., imatinib mesylate or related compound) to the drusen deposit through specific binding. Such antibodies are known and have been described. See, e.g., U.S. Patent Application Publication Nos. 2011/0020237 by Glabe et al. and 2009/0069258 by Chain, and U.S. Pat. No. 7,901,689 by Chain.

Combination therapy can be sequential therapy where the subject is treated first with one composition or drug and then the other, or alternatively, the two drugs can be given simultaneously and administered to the same or different sites in the same or different frequencies and amounts. If administered at different times, the time interval between the initial and/or subsequent administrations of each drug or therapy can be determined by the subject's physician. However, exemplary intervals can include 1 hour or more, 2 hours or more, 3 hours or more, 4 hours or more, 5 hours or more, 6 hours or more, 7 hours or more, 8 hours or more, 9 hours or more, 10 hours or more, 12 hours or more, 24 hours or more, 2 days or more 5 days or more, and 7 days or more, between the administration of the first and second drugs or therapies.

Certain embodiments of methods and compositions provided herein are further illustrated by the following Examples, which are provided for illustrative purposes only. The Examples are not to be construed as limiting the scope or content of the invention in any way.

### EXAMPLES

### Example 1

### Retinal Pigment Epithelial Stem Cell (RPESC)-Based Model of Drusen Formation

This example describes experiments showing that the expression of various drusen-related proteins, and genes encoding them, is unregulated in RPE derived from RPESC, after exposing the RPE to oxidative stress in vivo. The results show that RPE derived from such stem cells can be used as an in vivo model for drusen formation, e.g., to indentify compounds that modulate drusen formation.

More specifically, retinal pigment epithelial (RPE) cells are derived from RPE stem cells (RPESCs) and cultured using methods described, e.g., by Salero et al. (Cell Stem Cell (2011) 10:88-95) and/or in U.S. patent application publication No. 2009/0274667. Briefly, human RPE cells are dissected from cadaveric human eyes and cultured in RPE-THT media (DMEM/F12, 1x THT, 1% L-glutamine, 1% penicillin/streptomycin, 1% N1 supplement) from Sigma-Aldrich (St. Louis, Mo.) containing 10% fetal bovine serum (FBS) tapered down to 5FBS for approximately 60 days or until confluent on a Falcon Primaria 24-well plate or on a placental extracellular matrix-coated transwell membrane. Once confluent, the passage zero RPE are split into wells to be used for experiments, and again tapered from 10% to 5% FBS in RPE-THT media. The RPE are then allowed to grow until transepithelial resistance (measured weekly using a WPI Voltohmeter) is greater than 200 Ω·cm². The RPE are then transferred into RPE Classic media (DMEM/F12, 1% sodium pyruvate, 1% L-glutamine, 1% penicillin/streptomycin) with 5% FBS for the remainder of the experiment(s). Primary hRPE are passage 1-3 for the experiments described here.

Chronic oxidative stress may be induced following the protocol described by Glotin et al. (Free Radic. Biol. Med. (2008) 44:1348-136), adapted and modified as described herein. Specifically, RPE cells in confluent wells are fed with RPE Classic media with 5% FBS and containing between 500 μM and 2 mM tert-butyl hydroperoxide (TBHP) or H₂O₂ (to induce oxidative stress) or vehicle (for controls) for two hours each day over a period of five consecutive days, and then rested in RPE Classic media for 24 hours.

RNA is isolated from the RPE cells at the end of the 24 hour rest period, according to the protocol in the Qiagen RNeasy Micro/Mini Kits. Once isolated, the RNA is converted into cDNA using a high-capacity RNA to cDNA conversion kit (Applied Biosystems). The resulting cDNA is then used in a qPCR assay, to quantitate expression of various drusen-associated genes, including genes encoding: αA-crystallin, αB-crystallin, βB1-crystallin, βB2-crystallin, βs-crystallin, αA4-crystallin, APP, APOE, APOJ, BACE-1, PS1, VEGF, VEGF R1, VEGF R2, PEDF, CC9, serum amyloid P, TIMP3, vitronectin, and H2AE. The expression level of one or more housekeeping genes, such as ribosomal S18 and GAPDH, is also quantitated. Forward and reverse PCR primers having the nucleotide sequences shown in Table 2, below, may be used with a SYBR green reporter from Applied Biosystems.

TABLE 2
Primer Sequences for Detecting Drusen-Associated Protein Expression

| | | |
|---|---|---|
| mRNA Transcript | Forward Primer (5′-3′) | Reverse Primer (5′-3′) |

Crystallins

| | | |
|---|---|---|
| aA-crystallin | GGTGTCTGTCTTCCTTTGCTTCCCTT | TAAGCTCTCCTGGCTGCTCTCT |
| | (SEQ ID NO: 41) | (SEQ ID NO: 42) |
| aB-crystallin | AGGTGTTGGGAGATGTGATTGAGGTG | ACAGGGATGAAGTAATGGTGAGAGGG |
| | (SEQ ID NO: 43) | (SEQ ID NO: 44) |
| βB1-crystallin | CAGGAACATCCCCTAGTCCC | GTCTGCCAGATTTGACGACTC |
| | (SEQ ID NO: 45) | (SEQ ID NO: 46) |
| βB2-crystallin | AAGGGAGAGCAGTTTGTGTTTGAG | CTTGTGCTCTTGGCTGTCCACTTT |
| | (SEQ ID NO: 47) | (SEQ ID NO: 48) |
| βs-crystallin | GTATGAAACCACCGAAGATTGCCCTTCC | AGACACCCTCCAGCACCTTACA |
| | (SEQ ID NO: 49) | (SEQ ID NO: 50) |
| aA4-crystallin | ATGGGATGGGAAGGCAATGAAGTAGG | CCGGAAATGTTTGTAGTCACCGGAATG |
| | (SEQ ID NO: 51) | (SEQ ID NO: 52) |

Amyloid-β Related Proteins

| | | |
|---|---|---|
| APP | CCGCTGCTTAGTTGGTGAGTTTGT | ACGGTGTGCCAGTGAAGATGAGTT |
| | (SEQ ID NO: 53) | (SEQ ID NO: 54) |
| APOE | AACTGGCACTGGGTCGCTTT | GCCTTCAACTCCTTCATGGTCTCGT |
| | (SEQ ID NO: 55) | (SEQ ID NO: 56) |
| APOJ | ATTTATGGAGACCGTGGCGGAGAAAG | CTGGTTACTTGGTGACGTGCAGAG |
| | (SEQ ID NO: 57) | (SEQ ID NO: 58) |
| BALE-1 | CAGACAAGTTCTTCATCAACGGCTCCAAC | TTGGGAACGTGGGTCTGCTTTAC |
| | (SEQ ID NO: 59) | (SEQ ID NO: 60) |
| PS1 | GAGTTACCTGCACCGTTGTCCTACTT | TGTGCTCCTGCCGTTCTCTATTGT |
| | (SEQ ID NO: 61) | (SEQ ID NO: 62) |

Vascular Proteins

| | | |
|---|---|---|
| VEGF A | TCTTCAAGCCATCCTGTGTG | ATCCGCATAATCTGCATGGT |
| | (SEQ ID NO: 63) | (SEQ ID NO: 64) |
| VEGF R1 | TCTGGGACAGTAGAAAGGGCTTCATC | ACTGGGCGTGGTGTGCTTATTT |
| | (SEQ ID NO: 65) | (SEQ ID NO: 66) |
| VEGR R2 | CCTCTGTGGGTTTGCCTAGTGTTTCT | CCCTTTGCTCACTGCCACTCTGATTATTG |
| | (SEQ ID NO: 67) | (SEQ ID NO: 68) |
| PEDF | GCCCTGGTGCTACTCCTCT | GCATCGAGACTATCGCTAATGAG |
| | (SEQ ID NO: 69) | (SEQ ID NO: 70) |

Inflammation-Related Proteins

| | | |
|---|---|---|
| CC9 | ACGAACAGCAGGCTATGGGATCAA | CACGTTCCAAGGTCTTCGGTAGTATGT |
| | (SEQ ID NO: 71) | (SEQ ID NO: 72) |
| Serum Amyloid P | AGACCTCAGTGGGAAGGTGTTTGT | CTTGGGTATTGTAGGAGAAGAGGCTGTAGG |
| | (SEQ ID NO: 73) | (SEQ ID NO: 74) |

Basement Membrane Proteins

| | | |
|---|---|---|
| TIMP3 | TTCCCTTTGCCCTTCTCCTCCAATAC | CCTTGAGTCTATCTGCTTGCTGCCTTT |
| | (SEQ ID NO: 75) | (SEQ ID NO: 76) |
| Vitronectin | TTTAGGCATCGCAACCGCAAAGG | GCCAGTCCATCCTGTAGTCATCATAGTT |
| | (SEQ ID NO: 77) | (SEQ ID NO: 78) |

Misc. Drusen Proteins

| | | |
|---|---|---|
| H2AE | TGCTGTTAGGAAGCCACTATGTCTGG | ACACGGCCAACTGGAAACTGAA |
| | (SEQ ID NO: 79) | (SEQ ID NO: 80) |

Housekeeping Genes

| | | |
|---|---|---|
| Ribosomal S18 | GATGGGCGGCGGAAAATAG | GCGTGGATTCTGCATAATGGT |
| | (SEQ ID NO: 81) | (SEQ ID NO: 82) |
| GAPDH | ACAGTCGCCGCATCTTCTT | ACGACCAAATCCGTTGACTC |
| | (SEQ ID NO: 83) | (SEQ ID NO: 84) |

Transcripts for the drusen-associated proteins αβ2-crystallin, Aβ, APP, APOE, APOJ, BACE-1, PS1, TMP3, CC9 and VEGF A were significantly upregulated in the RPE cells after oxidative stress, compared to controls treated with only vehicle. Increased expression of the αB-crystallin and Aβ proteins in those cells was confirmed by immunocytochemical staining.

### Example 2

### RPE Cell-based Screening Assay

This example describes experiments in which the RPESC-based model of drusen formation, exemplified in Example 1, supra, is used to identify compound that modulate (e.g., reduce or inhibit) drusen formation in vitro. Compounds identified in such assays are thus useful for modulating (e.g., reducing or inhibiting) the formation of drusen in vitro or in vivo, and may therefore be useful, e.g., in therapeutic compositions and methods for treating or inhibiting conditions associated with drusen formation, including AMD and, in particular, dry AMD.

Specifically, RPE cells are prepared from RPESC and treated with either TBHP or H₂O₂ (to induce oxidative stress) or vehicle (for control) as described in Example 1, above. To investigate a test compound's effect on drusen expression, RPESC are incubated in media containing test compound or vehicle (as a control) during treatment with H₂O₂ or TBHP as described in Example 1, supra.

In this example, the RPESC-derived RPE cells are incubated with either imatinib mesylate (1 μM), DAPT (1 μM), ponatinib (500 nM or 1 μM) or bosutinib (1 μM) as test compounds. These test compounds are inhibitors of either γ-secretase (imatinib mesylate and DAPT) or c-Abl tyrosine kinase (ponatinib and bosutinib). DAPT, in particular, is a specific inhibitor of many functions of γ-secretase, including APP/Notch. In contrast, imatinib mesylate has a broader range of actions: it inhibits γ-secretase cleavage of APP, but not Notch, by binding to and inhibiting the γ-secretase activating protein (GSAP). Imatinib mesylate also inhibits PDGFR, as well as c-Abl tyrosine kinase, and upregulates neprilysin, an enzyme that degrads Aβ.

After treatment with a test compound, the RPESC-derived RPE may be assayed for one or more of the following criteria: restored viability, restored resistance, and reduced drusen expression. Each of these criteria, which are described in detail below, is indicative of drusen formation and/or drusen-associated diseases such as “dry” and other forms of AMD.

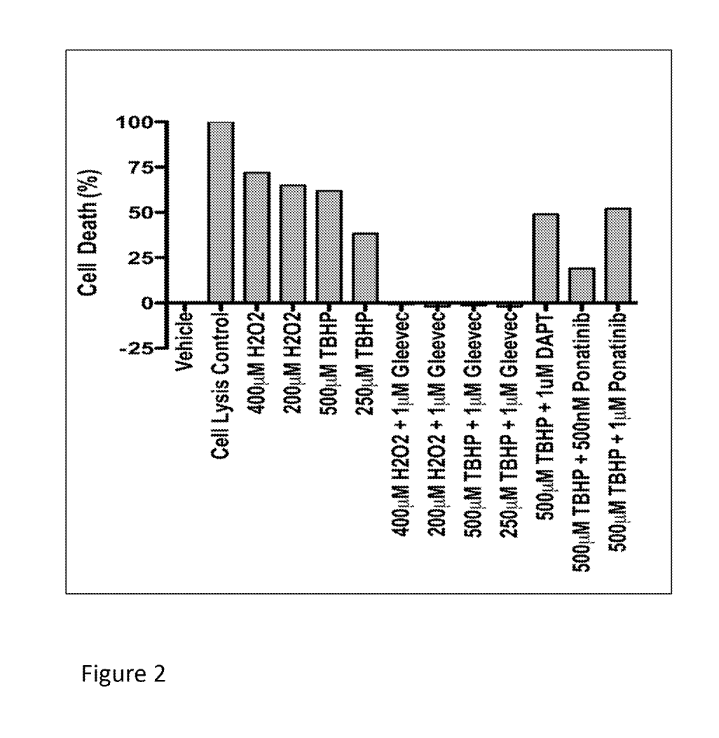

Restored viability. Briefly, viability of the stressed RPE following treatment with a test compound may be assayed by measuring lactate dehydrogenase levels in media, which is released by lysed or porous cells and accounts for both apoptosis and necrosis. More specifically, media from the RPE culture(s) is collected twenty-two (22) hours after inducing oxidative stress, and assayed using a Roche Cytotoxicity Detection Kit Plus (LDH) kit following the manufacturer's instructions. Treatment of the RPESC-derived RPE with 1 μM imatinib mesylate completely restores cells viability to 100% compared to untreated controls (i.e., 0% cell death in treated cells, compared to 100% cell death in untreated controls). As shown in FIG. 2, treatment with 1 μM DAPT and/or 1 μM ponatinib has no effect on cytotoxicity in this experiment. However, a beneficial effect is seen from treatment with just 500 nM ponatinib (about 80% reduction in cell death compared to the cell lysis control). DAPT does not attenuate cell death at in this experiment at any does tested between 0.1 μM and 10 μM. This suggests that c-Abl, which is inhibited by ponatinib but not by DAPT, is the target responsible for cell death in these experiments.

Restored resistance. Healthy RPE form tight functioned epithelia layers with high electrical resistance. Compromising the integrity of these layers decreases the resistance and is indicative of a diseased state, such as dry or other forms of AMD. Hence, an increased electrical resistance in treated RPE, compared to untreated cells, is another indicator that a test compound inhibits drusen formation and prevents or reduces a diseased state, such as AMD.

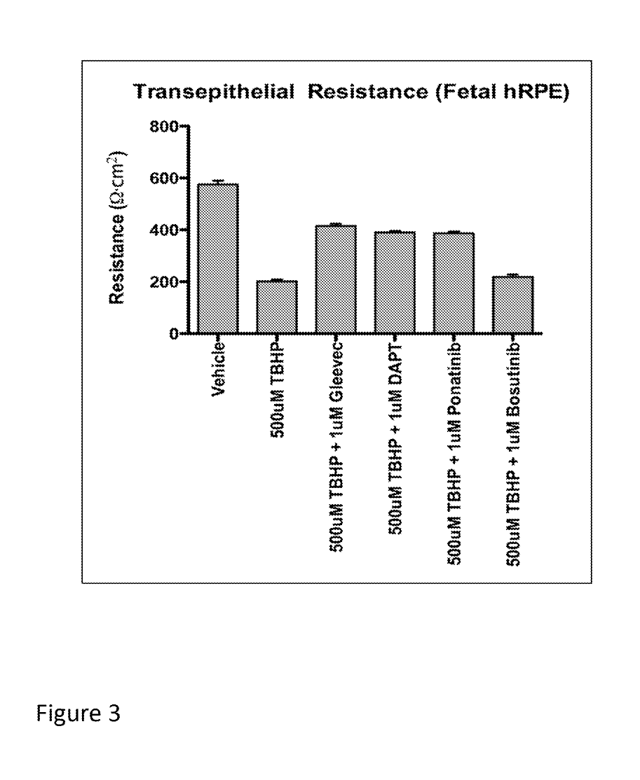

Transepithelial resistance is preferably measured prior to each treatment period, using the WPI EVOM Voltohmmeter protocol (Sheldon & Steinberg, Exp. Eye Res. (1977) 25(3):235-248; Li et al., Investigative Ophthal. (2007) 48:5722-5732), to confirm that cells in both treatment and control groups have similar or comparable levels of polarization. Preferably, each well of primary human RPE cells has a resistance greater than 200 Ω·cm² before beginning the experiment. Resistance of RPE treated with a test compound is measured again, after oxidative stress, and compared to the resistance in untreated RPE subjected to the same oxidative stress procedure. The results from such experiments are shown in FIG. 3. Imatinib mesylate, as well as DAPT and ponatinib, attenuate the decrease in transepithelial resistance observed upon after inducing oxidative stress by treatment with TBHP.

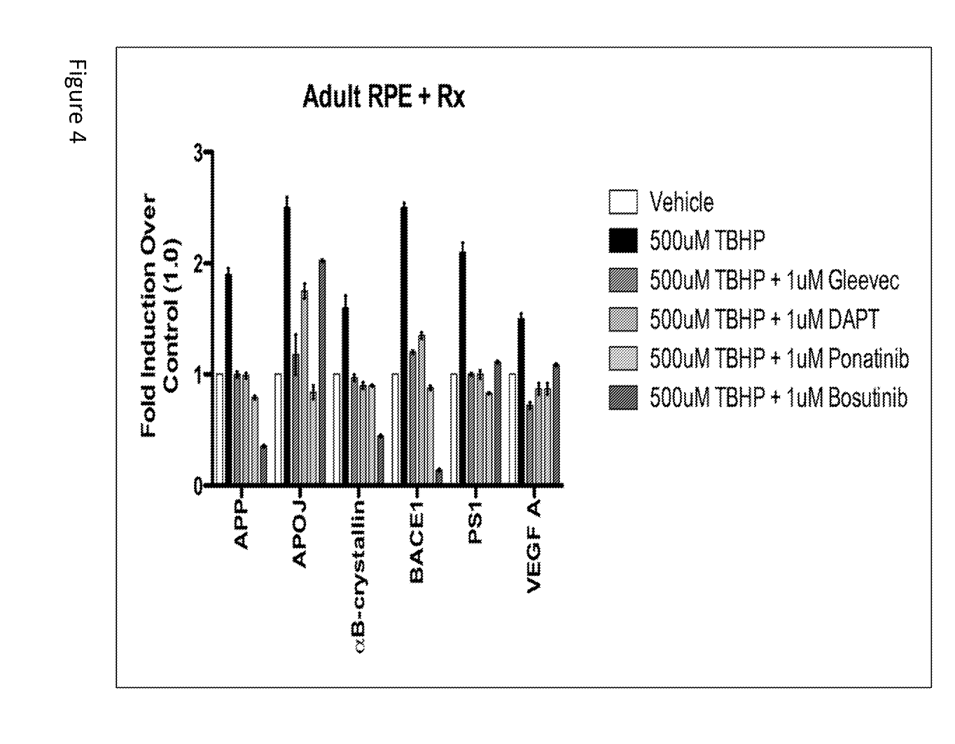

Reduced drusen expression. A test compound's ability to inhibit drusen formation may also be assayed by assaying expression levels of drusen-related proteins (or of genes encoding such proteins) as demonstrated, e.g., in Example 1 above, for both treated and untreated (control) cells. Results from such experiments are depicted in FIG. 4. Imatinib mesylate attenuates TBHP-induced up-regulation of drusen-associated protein transcripts, as do DAPT, ponatinib and bosutinib.

### Conclusion

The above-described experiments indicate pathways that inhibit amyloid beta (Aβ) synthesis and other mechanisms of imatinib action, which may contribute to the restoration of cell viability and function in imatinib mesylate treated RPE. In particular, compounds that inhibit AP production by more specific mechanisms thatn imatinib mesylate—including DAPT, ponatinib and bosutanib—are tested in the in vitro RPE-based model of drusen formation as described above. At 1 μM only imatinib mesylate restores all three of the criteria tested in the above experiments, suggesting that the compound may have a combined effect on multiple targets contributing to drusen formation and effecting epithelial integrity; such as kinase inhibition and/or decreased Aβ formation.

Out of the drugs tested in these experiments, imatinib mesylate is the most effective at restoring all three of the criteria investigated: RPE cell viability, epithelial resistance and the expression of drusen-related proteins.

### Example 3

### In Vivo Testing of Candidate Drusen Inhibitors

Test compounds, including imatinib mesylate and other compounds identified as candidate drusen inhibitors, e.g., in the in vitro assays demonstrated supra, may also be tested in vivo, using animal models of AMD. For example, Malek et al. (Proc. Natl. Acad. Sci. U.S.A. (2005) 102:11900-11905) describe a mouse model, referred to as the APOE4-HFC mouse of AMD that can be used to test compounds in vivo. Generally speaking, a candidate or test compound's effect on drusen formation can be tested by administering the compound to such as mouse (e.g., orally, intravenously, parentally or intraocularly) and its effect on drusen formation evaluated by comparing histological changes in the retina or RPE of treated and untreated mice.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. The method of claim 3, wherein the composition comprises imatinib mesylate.

2. A method of inhibiting drusen in a subject, which comprises administering to a subject in need thereof an effective amount for inhibiting drusen of a composition that: (i) inhibits gamma secretase; (ii) inhibits gamma secretase activating protein (GSAP), (iii) inhibits platelet derived growth factor receptor (PDGFR); (iv) inhibits c-Abl tyrosine kinase; (v) accelerates Aβ resorption; and/or (vi) upregulates neprilysin.

3. The method of claim 2, wherein the composition is a member selected from the group consisting of imatinib mesylate, ponatinib, bosutanib, dasatinib, PD 180970, sunitinib, DAPT, and bexarotene.

4. The method of claim 1, wherein the subject is suffering from or is at risk of developing dry age-related macular degeneration (AMD).

5. The method of claim 1, wherein the administration is intraocular, oral, or parental.

6. The method of claim 1, wherein the composition is administered daily at least once.

7. The method of claim 1, wherein the subject is one or more of a patient, a mammal, and a human.

8-9. (canceled)

10. The method of claim 1, wherein the method comprises decreasing the expression level of one or more drusen-related polypeptides.

11. The method of claim 10, wherein the one or more drusen-related polypeptides is a member selected from the group consisting of amyloid precursor protein (APP), apolipoprotein J (APOJ), apolipoprotein E (APOE), amyloid beta (Aβ), alphaB-crystallin, β-site AβPP cleaving enzyme 1 (BACE-1), presenilin 1 (PS1), and vascular endothelial growth factor (VEGF)-A.

12. The method of claim 14, wherein the composition comprises imatinib mesylate.

13. A method of treating drusen which comprises administering to a patient in need of such treatment an effective amount for treating drusen of a composition that: (i) inhibits one or more of: gamma secretase, (GSAP), (PDGFR), and c-Abl tyrosine kinase, and/or (ii) accelerates Aβ resorption.

14. The method of claim 13, wherein the composition comprises one or more of imatinib mesylate, ponatinib, bosutanib, dasatinib, PD180970, DAPT, and bexarotene.

15. A method of treating dry AMD which comprises administering to a subject in need of such treatment an effective amount for treating dry AMD of a composition that inhibits: (i) gamma secretase and/or (GSAP), (ii) (PDGFR), and (iii) c-Abl tyrosine kinase.

16. The method of claim 15, wherein the composition comprises one or more of imatinib mesylate, ponatinib, bosutanib, dasatinib, PD180970, DAPT, and bexarotene.

17. The method of claim 15, wherein the composition accelerates Aβ resorption.

18. The method of claim 2, wherein the subject is suffering from or is at risk of developing dry age-related macular degeneration.

19. The method of claim 2, wherein the administration is intraocular, oral or parental.

20. The method of claim 2, wherein the composition is administered daily at least once.

21. The method of claim 2, wherein the subject is one or more of a patient, a mammal, and a human.

22. The method of claim 2, wherein the method comprises decreasing the expression level of one or more drusen-related polypeptides.

23. The method of claim 22, wherein the one or more drusen-related polypeptides is a member selected from the group consisting of APP, APOJ, APOE, Aβ, alphaB -crystallin, BACE-1, PS1, and VEGF-A.
